SEQUENCE LISTING (3 sequences)

SEQ ID NO: 1 - protein, 545 aa, P. Alba
  INIT_MET (1)..(1)
  ref <308>: DATABASE ACCESSION NUMBER: D8UY75_POPAL
  ref <309>: DATABASE ENTRY DATE: 2010-10-05
  ref <313>: RELEVANT RESIDUES IN SEQ ID NO: (52)..(595)
MTEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 2 - DNA, 1656 bp, P. patens
  CDS (7)..(1650)
ggatccatgactgaagctagacgttctgctaattatgagcctaactcgtgggattacgattatctgttgtcgtccgataccgatgaatcaatcgaggtgtacaaggataaggctaagaaattggaggccgaagtgaggagagagattaataacgagaaggctgaattcttgacattgcttgagctgattgataacgtgcagcgattggggctcggctatcgttttgagagcgatatccgtggtgctcttgatcgctttgtgtcatcgggaggattcgacgccgttaccaagacgtctttgcacggaaccgccctgagcttcaggcttctgaggcaacacgggtttgaggtgagtcaagaggctttcagcggtttcaaagatcagaatggcaattttctggagaacttgaaggaggatattaaggcaattctctcactttacgaagctagtttccttgctctggaaggtgagaatattttggacgaagctaaggtgtttgctattagtcatctgaaggagctgtcagaggaaaagatcggtaaagaacttgccgaacaggtaaatcacgcactcgaactcccgctgcatcgtagaacgcagaggctcgaggctgtatggagtattgaggcataccgcaagaaggaagacgctaaccaggtcctgctggagctggcaatcctcgactataacatgattcaaagcgtgtaccaaagagatctccgcgaaacgtcacgttggtggcgtcgggtggggctcgccacaaagttgcatttcgcacgcgatcgacttattgaatctttctactgggccgtgggtgttgccttcgaaccgcagtactctgactgccggaattcggtggccaagatgtttagcttcgttaccattatcgacgatatttatgacgtttacggcacattggatgagctggaactgtttaccgacgccgtggaacgttgggacgttaacgcaatcaacgacctgcccgactatatgaaactctgcttcctggcactgtacaacactattaatgagatagcttacgataatctgaaggacaagggtgaaaacatcctcccatatctcaccaaggcttgggccgatctgtgtaacgcattcctccaagaggccaagtggctttacaataaatctaccccgacattcgatgactacttcgggaacgcatggaagagttcctcggggcccctccaacttgtgttcgcttatttcgctgttgtgcagaacattaagaaggaggagattgaaaaccttcaaaaataccacgatacgatttcgcgaccttcacatatctttaggctttgtaacgaccttgcatctgctagcgctgaaatcgctagaggcgagactgccaacagtgtgtcgtgctacatgaggactaagggtatcagcgaggagctcgcaaccgagtcagtgatgaacttgattgacgaaacttggaaaaagatgaacaaggaaaaattgggcggatccttgttcgctaagccgtttgtggagacagccattaatttggcacggcaatcccattgcacgtatcataatggagacgcacacacgagtccagatgaactgacgcgaaagcgggtactgagcgtgattactgagcctattctcccctttgagcggtagtagtagggatcc

SEQ ID NO: 3 - protein, 545 aa, P. patens; identical to SEQ ID NO: 1

(19) United States
(12) Patent Application Publication
(10) Pub. No.: US 2011/0262986 A1
(43) Pub. Date: Oct. 27, 2011

(54) BIOLOGICAL SOLAR PANEL DEVICES, ARRAYS, METHODS AND SYSTEMS FOR THE COLLECTION OF VOLATILE ORGANIC MOLECULES

(76) Inventor: Jonathan J. Burbaum, San Diego, CA (US)

(21) Appl. No.: 12/986,202

(22) Filed: Jan. 7, 2011

Related U.S. Application Data
(60) Provisional application No. 61/293,568, filed on Jan. 8, 2010.

(51) Int. Cl. C12P 5/02; C12M 1/00

(52) U.S. Cl. 435/167; 435/292.1

(57) ABSTRACT

Provided herein are devices, arrays, methods, systems, and other subject matter comprising a biological solar panel device comprising: (a) a layer comprising a material is transparent or translucent to light; (b) a photosynthetic layer comprising a material that uses carbon dioxide and water in the presence of sunlight to release a volatile organic molecule, wherein the photosynthetic layer is separated from the transparent or translucent material by a gas layer; and (c) a layer that provides support for the material that releases a volatile organic molecule.

### CROSS REFERENCE TO RELATED APPLICATIONS

This application claims the benefit of U.S. Provisional Patent Application Ser. No. 61/293,568 filed on Jan. 8, 2010.

### BACKGROUND OF THE INVENTION

Processes for converting biomass to chemical energy such as ethanol or organic molecule production have been explored. For example, much attention has been given to ethanol in particular, which has a lower energy content than gasoline such that more ethanol is required to provide the same energy output. More significantly, both ethanol and lipid productions are currently driven by fossil fuel. It has been well documented that the energy input for ethanol production may exceed the energy output from the combustion of ethanol.

### SUMMARY OF THE INVENTION

The subject matter provided herein utilizes biological solar panel technology. Biological solar panel technology captures and converts sunlight energy into chemical energy, in a form that can be isolated, stored, and transported more efficiently than other forms of energy and methods, systems, and devices known in the art. This innovative field of endeavor, referred to as chemiculture, harnesses the power of photosynthesis to provide useful chemical products in an energy-efficient process. Provided herein are devices, methods, systems, and other subject matter that address shortcomings in the art for converting solar energy to chemical energy. Further provided herein is a superior approach to alternative energy creation.

In certain embodiments, the subject matter provided herein utilizes the DOXP shunt pathway and other similar natural and non-natural processes. The DOXP pathway, for example, creates organic molecules, including isoprene (isopentadiene), from carbon dioxide (CO₂) and water in the presence of sunlight as illustrated in FIG. 8. In particular, isopentenyl pyrophosphate (IPP) and dimethylallyl pyrophosphate (DMAPP) are the building blocks of various volatile molecules found naturally in plant material, classified broadly as terpenes. As further depicted in FIG. 8, the MVA pathway is also an entry point to diterpene biosynthesis via dimethylallyl trans transferase production of geranyl diphosphate.

The DOXP pathway is an alternative pathway for carbon fixation that provides a bypass to glucose biosynthesis. This pathway is active in a number of photosynthetic organisms including, for example, oak (where it has been implicated in air pollution) and the rapidly growing and the resilient vine/weed, kudzu. From an evolutionary perspective, isoprene may confer thermotolerance to photosynthetic organisms, and is induced under environmental stress. For example, in certain situations, isoprene is induced under water deprivation as well as elevated temperatures, and in some instances accounting for more than 60% of all photosynthetic activity.

The DOXP pathway is more efficient in capturing the sun's energy than ethanol production from biomass. Ethanol production requires a second organism (generally, the yeast Saccharomyces cerevisiae), growing under anaerobic conditions. In contrast, synthesis of isoprene can be performed in a single organism. What is more, isoprene has almost 50% higher energy content than ethanol (44.1 MJ/kg for isoprene, vs. 29.7 MJ/kg for ethanol).

In one aspect, provided herein are methods, devices and systems comprising a biological solar panel device comprising: (a) a layer comprising a material that is transparent or translucent to light; (b) a photosynthetic layer comprising a material that uses carbon dioxide and water in the presence of sunlight to release a volatile organic molecule, wherein the photosynthetic layer is separated from the transparent or translucent material by a gas layer; and (c) a layer that provides support for the material that releases a volatile organic molecule. In some embodiments, more than one of the devices described herein are connected to form an array.

In another aspect, provided herein is a biological solar panel device comprising a transparent or translucent layer. In another aspect, provided herein is a biological solar panel device comprising a material that uses carbon dioxide and water in the presence of sunlight to release a volatile organic molecule that comprises at least one photosynthetic organism that is living. In another aspect, provided herein is a biological solar panel device comprising a gas layer. In another aspect, provided herein is a biological solar panel device comprising a support layer.

In yet another aspect, provided herein is a biological solar panel comprising a photosynthetic organism or photosynthetic-like organism that is a member of the Plantae kingdom. In another aspect, provided herein is a biological solar panel device comprising a photosynthetic-like organism and the photosynthetic-like organism is a member of the Fungi kingdom. In another aspect, the photosynthetic or photosynthetic-like organism is living and is a member of the Monerans kingdom.

In another aspect of the subject matter described herein, the material that uses carbon dioxide and water in the presence of sunlight to release a volatile organic molecule comprises a non-living material.

In another aspect, provided herein is subject matter that harnesses organisms that naturally produce volatile organic molecules, including for example the organic molecule isoprene. Another aspect of the subject matter described herein is an organism that has been genetically modified to produce increased levels of a volatile organic molecule of interest.

In another aspect of the subject matter provided herein, a material or organism of a biological solar panel device produces a volatile organic molecule that comprises at least one terpene. In yet an additional aspect, provided herein is a device herein comprises a volatile organic molecule that comprises at least one terpenoid.

Another aspect provided herein is a method of manufacturing a biological solar panel device. In yet another aspect, provided herein are methods, devices and systems comprising collecting isoprene that has been released from a living or non-living material that is in a photosynthetic layer of a biological solar panel device. In a further aspect, provided herein is a method, device and system for the collection of a volatile organic molecule using hollow fiber gas concentration.

In an additional aspect, provided herein are certain genetically modified organisms.

### BRIEF DESCRIPTION OF THE DRAWINGS

Many features herein are set forth with particularity in the appended claims. A better understanding of the features and advantages herein will be obtained by reference to the following detailed description that sets forth illustrative embodiments, in which many principles are utilized, and the accompanying drawings of which:

FIG. 1 is illustrative of a biological solar panel device.

FIG. 2 is illustrative of an exemplary transparent or translucent layer as described herein.

FIG. 3 is illustrative of a photosynthetic layer of a device provided herein.

FIG. 4 is illustrative of a biological solar panel array.

FIG. 5 is illustrative of a biological solar panel displayed in natural sunlight.

FIG. 6 is illustrative of an aspect of a hollow fiber collection unit.

FIG. 7 is illustrative of an aspect of a hollow fiber collection unit.

FIG. 8 is illustrative of the DOXP biosynthetic pathway that leads to isoprene formation in certain photosynthetic organisms.

FIG. 9 is illustrative of an exemplary method of plasmid construction and genetic modification of moss.

### DETAILED DESCRIPTION OF THE INVENTION

The biological solar panel devices, arrays, methods, systems, and other subject matter provided herein capture energy from sunlight, which is then converted to fuel potential. While preferred embodiments of the present invention have been shown and described herein, a person of ordinary skill in the art will appreciate that such embodiments are provided by way of example only. Numerous variations, changes, and substitutions will now occur to those skilled in the art without departing from the invention. It should be understood that various alternatives to the embodiments of the invention described herein are employed in practicing the invention. Many features herein are set forth with particularity in the appended claims. It is intended that the claims define the scope of the invention and that methods and structures within the scope of these claims and their equivalents be covered thereby.

A better understanding of the features and advantages of the present invention will be obtained by reference to the following detailed description that sets forth illustrative embodiments, in which many principles of the invention are utilized. The section headings used herein are for organizational purposes only and are not to be construed as limiting the subject matter described.

### Biological Solar Panel Devices

In one aspect, provided herein is a biological solar panel device that comprises: a layer comprising a material that is transparent or translucent to light; a photosynthetic layer comprising a material that uses carbon dioxide and water in the presence of sunlight to release a volatile organic molecule, wherein the photosynthetic layer is separated from the transparent or translucent material by a gas layer; and a layer that provides support for the material that releases a volatile organic molecule. In some embodiments, the device comprises a photosynthetic biomaterial that emits a volatile organic compound in a form that can be collected from the vapor phase. In certain embodiments, the devices provided herein are essentially planar collection devices that are manufactured or formed into a sheet that is much thinner than its length and width. For example in certain instances, the devices are thin enough to have a small volume, but are thick enough to capture incident light efficiently. In these situations, because of the small volume of the device relative to its surface area for light collection, the volatile molecule is generated in a concentrated form (compared to the more three dimensional growth of, for example, trees). This reduced volume enables the efficient collection of volatile molecules emitted from vegetation by photosynthesis.

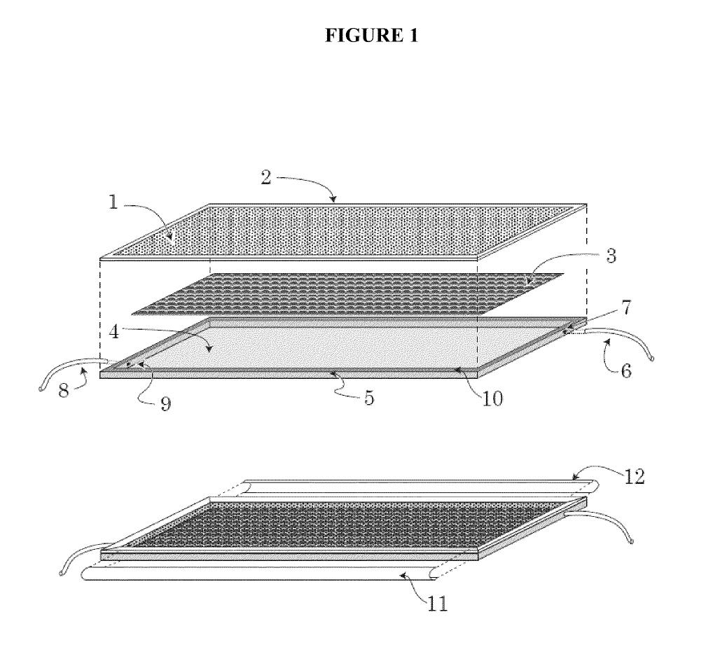

FIG. 1 demonstrates an exemplary, non-limiting embodiment of the subject matter described herein. Referring to FIG. 1, a transparent or translucent layer 1 is comprised of a frame 2 and gas a permeable membrane 1. A photosynthetic layer 3 is affixed above a support layer 4 with a frame 5. Fluid access (gas and liquid) is provided by inlet and outlet ports 7 and 9, which are connected to inlet and outlet hoses 6 and 8. A seal is provided between the layers through gasket 10 and side clamps 11 and 12.

In some embodiments, the dimensions of the device are about 4 ft by about 8 ft, with a gap separating the layers of about 0.5 inches. In other embodiments, a single device is between about 1 inch to about 100 ft in width and about 1 in to about 100 ft in length. In some embodiments, the gap separating the layers is about 0.05 in to about 12 inches. It is contemplated that the several (at least two) devices will be connected, forming what is referred to herein as an array.

In an exemplary embodiment, a device herein comprises a photosynthetic inner layer affixed between two outer layers. In certain situations, at least one of the layers is phototransparent and optionally blocks harmful UV radiation that can accelerate the decomposition of isoprene (via hydroxyl, hydroperoxyl, and ozone radicals).

In some instances, the layers are mostly or completely impermeable to the volatile organic molecule that is being generated. In other instances, one or more of the layers absorbs the molecule to facilitate isolation.

In some situations, the biological solar panel devices provided herein included biological solar panel arrays are displayed in natural sunlight. In further or additional embodiments, the devices and arrays provided herein are displayed in non-natural light.

In still further embodiments, the devices provided herein provided a fully-enclosed system that prevents the release of photosynthetic organisms (including, for example, genetically-modified organisms) into the environment. In additional embodiments, the device is accessible for fluids and gases through ports on one or both ends of the device. These ports allow for the introduction of nutrients, gases, liquids, and the like (including carbon dioxide) and the removal of terpenes, oxygen, and other waste products.

In yet an additional embodiment, a device or system provided herein operates in continuous flow mode (with constant flow of gases and volatiles in and out) or as a batch device (where gases are exchanged and volatiles are harvested periodically), or combination thereof.

In some embodiments, a layer is optionally recyclable. A recyclable layer allows for replacement of the photosynthetic layer when it is no longer viable, or when an improved photosynthetic layer becomes available.

Transparent of Translucent Layer

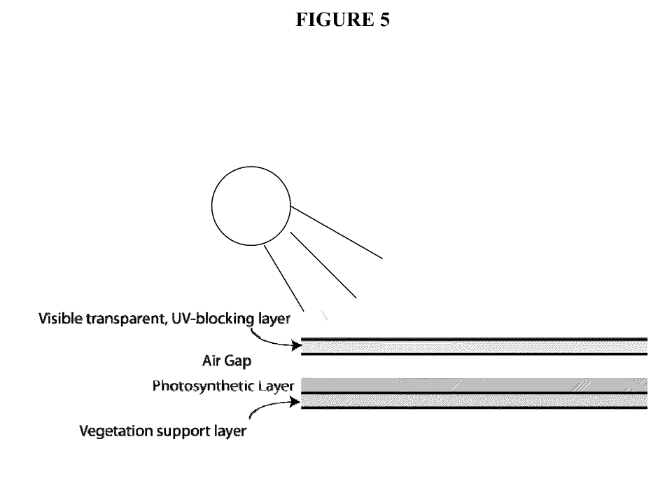

In another aspect, provided herein is a biological solar panel device comprising a transparent or translucent layer. For example, in certain embodiments, the transparent or translucent layer of the subject matter provided herein comprises a solid material. See, e.g., FIG. 5. In certain instances, the transparent or translucent material comprises a polymeric material. In some instances, the polymeric material comprises an organic polymer. In some instances, the transparent or translucent material comprises a polysilicate material.

In some instances, the transparent or translucent material of the device comprises a gas permeable material. In some instances, the transparent or translucent layer is selectively permeable to at least one oxygen-containing molecule flowing into the photosynthetic layer. In some instances, the at least one oxygen-containing molecule is carbon dioxide.

In some instances, the transparent or translucent layer is substantially impermeable to carbon dioxide flowing from the photosynthetic layer past the transparent or translucent layer. In some instances, the transparent or translucent layer is substantially impermeable to any volatile organic molecule. In some instances, the transparent or translucent material is substantially impermeable to at least one volatile organic molecule. In another instance, the transparent or translucent layer reversibly adsorbs the volatile organic molecule.

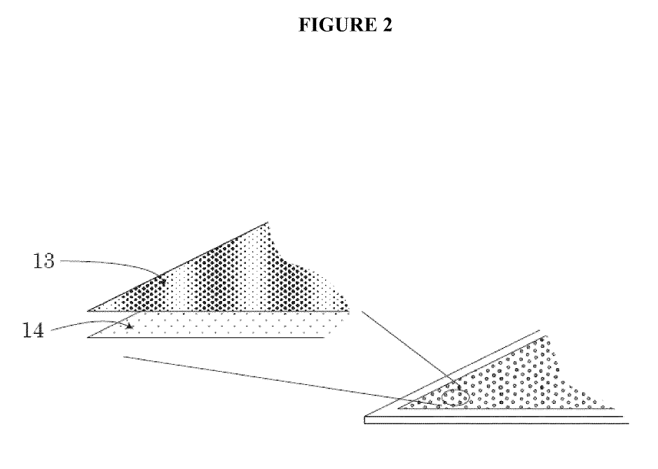

FIG. 2 represents an exemplary transparent or translucent layer as described herein. In this example, the gas permeable membrane is comprised of two surfaces, both of which transmit visible light. The outer surface 13 transmits carbon dioxide but protects against dehydration, and thus in certain situations is hydrophobic and UV absorbing, for example with a low-density polyethylene film (LDPE) that is UV stabilized. For example, in some embodiments, the transparent or translucent material comprises a biaxially-oriented polypropylene that is UV stabilized, e.g., using TU BOPP UV-stabilized general purpose film that is produced by the AmTopp division of the Inteplast Group, ltd. The inner surface 14 also transmits carbon dioxide but is not porous to non-polar gases such as isoprene. In certain situations, the surface is hydrophilic and charged, for example with 3M Anti-Fog Polyester Film 9971. In some situations, the membrane is mounted in an aluminum frame such as one that would be used in window screens, for example Prime-Line PL-14201 affixed with a retainer spline.

In some instances, the transparent or translucent layer material of the device blocks at least some light in the ultraviolet spectrum. In further or additional embodiments, the transparent or translucent layer material blocks all of the light in the UV spectrum. In some instances, the transparent or translucent layer material blocks some of the light in the visible spectrum. Still, in further embodiments, the transparent or translucent layer material blocks at least some or all of the light in the infrared spectrum or higher wavelength spectrums.

Photosynthetic Layer

Another aspect of the subject matter described herein, the material that uses carbon dioxide and water in the presence of sunlight to release a volatile organic molecule comprises a non-living material. For example, in some situations the non-living material mimics the biological processes described herein, including for example photosynthesis generally, or in other situations, the DOXP pathway in particular. In some instances, the material that uses carbon dioxide and water in the presence of sunlight to release a volatile organic molecule comprises at least one photosynthetic organism that was or is living. In some instances, the material that uses carbon dioxide and water in the presence of sunlight to release a volatile organic molecule comprises at least one photosynthetic organism that is living. See, e.g., FIG. 5.

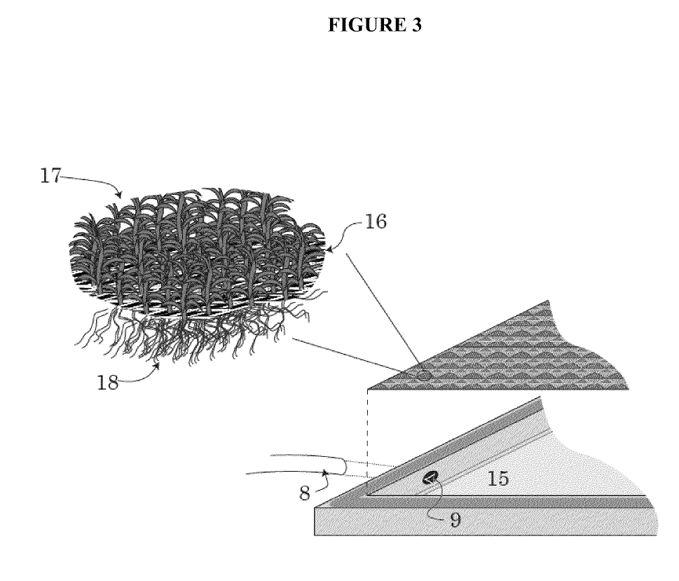

FIG. 3 demonstrates an exemplary and non-limiting embodiment of an example of a photosynthetic layer of a device herein. In this example, the photosynthetic layer is grown in situ. For example, Physcomitrella patens is transformed with a gene that expresses an unregulated, catalytically-active form of isoprene synthase (IspS) (vide infra). To construct this layer, protonema cells are propagated in liquid culture are seeded onto layer 15, which contains Knop medium (Reski and Abel, 1985), comprised of the following stock solutions: 25 g/l KH₂PO₄, 25 g/l KCl, 25 g/l MgSO₄×7H₂O, 100 g/l Ca(NO₃)₂. One liter of Knop medium contains 10 ml of each stock solution with 12.5 mg FeSO₄×7H₂O is adjusted to pH 5.8 with KOH or HCl. This is then incorporated into an 6.5-0.8% agarose matrix. After the cells begin to propagate (1-3 days), the protonema layer is then overlaid with fine mesh netting 16 (for example American Home & Habitat 0.25 mm polyester netting FFM010). Upon growth to a integrated layer, the photosynthetic layer is comprised of a photosynthetic layer 17 and a protonema layer 18, which is embedded into the agarose layer, which provides essential nutrients and water.

In some embodiments, the photosynthetic layer provided herein reaches a static phase where further horizontal and vertical growth are inhibited. In this way, production of the volatile product allows the photosynthetic layer to remain intact and reusable for the biological lifetime of the plant material. In some embodiments, the photosynthetic layer and/or biological materials do not need to be processed in order to isolate the volatile material.

### Members of the Plant Kingdom

In another aspect, provided herein is a biological solar panel device comprising a photosynthetic organism and the photosynthetic organism is a member of the Plantae kingdom. In some instances, the organism is a member a member of the Anthocerotophyta division. In some instances, the organism is a member of the Bryophyta division, and in further or additional instances, the organism is a member of the Musci subdivision. In some instances, the organism is a member of the Andreaeopsida class. In some instances, the organism is a member of the Bryopsida class, and in further or additional instances, the organism is a member of the Bryidea subclass. In some instances, the organism is a member of the Funariales order. In some instances, the organism is a member of the Funariaceae, family. In some instances, the organism is a member of the Physcomitrella Bruch & Schimp genus.

In some instances, the organism is Physcomitrella patens.

In some instances, the organism is a member of the Dicranales order. In some instances, the organism is a member of the Ditrichaceae family. In some instances, the organism is a member of the Ceratodon Brid. genus.

In some instances, the organism is Ceratodon purpureus.

In some instances, the organism is a member of the Sphagnopsida class.

In some embodiments, an organism of the device is selected from the taxonomic division selected from the group consisting of Charophyta, Chlorophyta, Chrysophyta, Craspedophyta, Cryptophycophyta, Euglenophycota, Haptophyta, Hepatophyta, Phaeophyta, Prasinophyta, Pyrrophycophyta, Pyrrophycophyta, Rhodophyta, and Xanthophyta.

In some embodiments, an organism of the device is selected from the taxonomic Chromista subkingdom. In some embodiments, an organism of the device is selected from the taxonomic Tracheobionta subkingdom.

In some embodiments, an organism of the device is selected from the taxonomic Magnoliophyta division. In some instances, the organism from the Magnoliophyta division as provided herein is a member of the Magnoliopsida class, and in some additional instances, a member of the Rosidae subclass. In some embodiments, the organism is a member of the Fabales order. In some embodiments, the organism is a member of the Fabaceae family. In some embodiments, the organism is a member of the Pueraria genus.

In some embodiments, the organism is kudzu.

In some embodiments, the organism is a member of the Hamamelidae subclass. In some embodiments, the organism is a member of the Fagales order. In some embodiments, the organism is a member of the Fagaceae family. In some embodiments, the organism is a member of the Quercus L. genus.

In some embodiments, the organism is a member of the Quercus alba L. species.

In some instances, the device herein comprises an organism selected from the group consisting of the following, without limitation: Abies alba (silver/European fir); Abies cephalonica (Greek fir); Abies cilicica (Turkish fir); Abies insignis; Abies marocana (Morocco fir); Abies numidica (Algerian fir); Abies pardei; Abies pinsapo (Spanish fir); Abies spp. (fir); Acacia nigrescens (African akazia); Acacia spp. (acacia); Acacia tortilis; Acer campestre; Acer platanoides (Norway maple); Acer spp.; Acmena smithii (lilly-pilly tree); Adenocarpus decorticans; Adiantum capillus-veneris; Aesculus flava (yellow buckeye); Afrormosia laxiflora; Afrostyrax lepidophyllus; Agelaea dewevrei; Agonis flexuosa (willow myrtle); Agrostis curtissii; Albizia adianthifolia; Albizia ferruginea; Albizia julibrissin (silk tree); Alnus rubra (red alder); Alnus sp.; Aloysia gratissima; Alsophila cooperi (cooper tree fern); Amelanchier alnifolia (serviceberry); Amorpha californica (California false indigo); Amorpha fruticosa (Iidigo bush); Ancistrophyllum secondiflorum; Angelea dewevrei; Arachis glabrata (peanut); Arachis hypogaea; Arbutus unedo (strawberry tree); Arctostaphylos glauca (bigberry manzanita); Arecastrum romanzoffianum (queens palm); Artemisia tridentata (basin/big sagebrush); Artocarpus falcata; Artocarpus heterophyllus; Arundinaria alpina; Arundo donax (reed grass/giant reed); Astartea fascicularis; Atrichum undulatum; Atriplex canescens (saltbush); Avena sativa “Dula” (oat); Avena spp., Bromus spp. (annual grassland); Avicennia spp.; Azolla sp.; B. t. var. atropurpurea (red barberry); Baccharis texana; Baeckea virgata; Bambusa multiplex (bamboo); Bambusa spp. (bamboo); Bambusa vulgaris (bamboo); Banksia ashbyi; Banksia laricina; Baphia nitida; Berberis aquafolium; Berberis dictyota (California barberry); Berberis nervosa (Cascade mahonia); Berberis nevinii (nevin barberry); Berberis pinnata (shinyleaf barberry); Berberis thungbergii (green barberry); Berberis trifoliata; Berlinia bracteosa; Berlinia confusa; Berlinia grandifolia; Betula lutea (yellow birch); Betula papyrifera (paper birch); Betula pubescens (European birch); Betula sp. (birch); Betula spp. (birch); Blighia sp.; Bocconia frutescens (plume poppy); Bouteloua rigideseta; Brotherella recurvans; Broussonetia spp. (paper mulberry); Burkea africana (red syringa); Bursera simaruba; Buxus sempervirens (dwarf boxwood); Buxus sempervirens (June) (dwarf boxwood); Buxus sempervirens (October) (dwarf boxwood); Callistemon citrinus (bottlebrush); Capparis cynophollophora (Jamaican Caper Tree); Capparis indica (Indian caper); Caragana arborescens (pea tree); Caragana maximowicziana; Caragana pekinensis; Carissa macrocarpa (Natal Plum); Carludovica insignis (hat palm); Carludovica palmata (Panama hat palm); Carpinus sp. (hornbeam); Carpinus spp. (hornbeam); Carya spp. (hickory); Castanea dentata (American chestnut); Casuarina (Australian pine); Casuarina cunninghamiana (beefwood); Casuarina equisetifolia (horsetail beefwood); Casuarina spp.; Catalpa spp.; Ceanothus crassifolius (hoaryleaf ceanothus); Cedrus spp.; Celtis spp.; Cercis canadenis (eastern redbud); Cercis spp. (redbud); Cercocarpus montanus (true mountain mahogany); Chamaecyparis spp.; Chamaerops humilis (Mediterranean fan palm); Chamaespartium tridentatum; Chrysothamnus nauseosus (rubber rabbit brush); Chusquea spp. (giant chusqua); Citrus limon (meyer lemon); Citrus sp.; Citrus spp.; Cladrastis lutea (yellow wood); Cladrastis platycarpa; Clusia rosea; Cocos nucifera (coconut palm); Colophospermum mopane (mopane); Colubrina taxensis; Cornbreturn apiculatum (myrobolan family); Combreturn molle (myrobolan family); Condalia hookeri; Conigramme intermedia; Cornus spp.; Corylus avellana (hazel (European)); Cotoneaster pannosus (cotoneaster); Crataegus spp. (hawthorn); Cupania anacardioides (carrotwood); Cupressus or Chamaecyparis (Evergreen cypress); Cyathea medullaris (sago fern); Cyperus spp.; Cyrilla spp. (red titi); Cytisus battandieri (Atlas broom); Cytisus multiflorus; Cytisus praecox (Scots broom); Cytisus scoparius; Cytisus sp.; Dalhousiea africana; Daniellia oliveri; Daphne gnidium; Dendromecon harfordii (island tree poppy); Dendromecon rigida (bush poppy); Dendromecon spp. (bush poppy); Detarium microcarpum; Dicksonia antarctica (tree fern); Dicksonia squarrosa (tree fern); Dicranum polyseturn; Diospyros spp.; Dryandra calophylla; Dryopteris filix-mas (fern); Elaeis guineensis (Palm oil tree); Embothrium coccineum (Chilean firebush); Ephedra californica; Ephedra chocuma; Ephedra nevadensis; Ephedra trifurca; Ephedra viridis; Erica arborea (tree heath); Erica arborea var. alpina (alpine heath); Erica australis; Erica carnea (spring heath); Erica ciliaris; Erica cinerea rose; Erica cineria white; Erica multiflora (heath); Erica umbellata; Eriogonum fasciculatum (California buckwheat); Eschscholzia californica (California poppy); Eucalyptus camaldulensis (red gum); Eucalyptus glaucescens; Eucalyptus globulus (blue gum); Eucalyptus globulus (adult) (blue gum); Eucalyptus globulus (young) (blue gum); Eucalyptus macrocarpa (desert rose); Eucalyptus maculata (spotted gum); Eucalyptus nicholii (willow leaved peppermint eucalyptus); Eucalyptus niphophilia (snow gum); Eucalyptus platypus (maalok); Eucalyptus polyanthemos (silver dollar gum); Eucalyptus pyriformis (pearpod mallee); Eucalyptus spp. (eucalyptus); Eucalyptus spp. (old) (eucalyptus); Eucalyptus spp. (young) (eucalyptus); Eucalyptus torquata (coral gum); Eucalyptus viminalis (ribbon gum); Eugema malacensis; Eugenia foetida; Eugenia grandis (eugenia); Eugenia uniflora (Barbados cherry); Eugenia xerophytica; Eysenhardtia taxana; Fagara heitzii; Fagus spp. (beech); Fagus syvatica (European beech); Feijoa sellowiana (pineapple guava); Ficus benjamina (Benjamin fig); Ficus capensis; Ficus carica (edible common fig); Ficus costaricana (higo fig); Ficus elastica (Indian rubber plant); Ficus fistulosa (Fig); Ficus glumosa; Ficus lyrata (fiddle-leaf fig); Ficus pumila (creeping fig); Ficus religiosa (religious fig); Ficus sp.; Frangula alnus Miller; Fraxinus sp.; Fraxinus spp. (ash); Fraxinus uhdei (evergreen ash); Funtumia elastica; Garcinia kola; Genista cinerea (ashy woodwaxen); Genista germanica (German woodwaxen); Genista scorpius (June); Genista scorpius (October); Gilbertiodendron dewevrei; Ginkgo biloba (ginkgo); Gleditsia triacanthos (common honey locust); Gliricidia sepum; Glycine max (soy bean); Gossypium hirsutum (cotton); Grevillea robusta (silk oak); Grevillea rosmarinifolia (rosemary grevillea); Grewia flavescens (grewia); Guibourtia demeusei; Hakea oleifolia (oliveleaf hakea); Hakea suaveolens (seet hakea); Hamamelis jelena; Hamamelis virginiana (common witch hazel); Harungana madagascariensis; Haumania danckelmaniana (f); Helianthus annuus (sunflower); Hevea brasiliensis (Rubber Tree); Hordeum vulgare (spring barley (Prisma)); Howea forsteriana (sentry palm); Hylocomium splendens; Hypericum calycinum (Aaronsbeard); Hypericum kalmianum (kalm St. Johnswort); Hypericum lanceolatum; Hypericum spathulatum; Ilex spp. (holly); Ingo spectabilis; Ipomoea mauritana; Irvingia gabonensis; Irvingia grandifolia; Irvingia smithii; Irvingia spp & Klainedoxa; Isoberlinia doka; Jubaea chilensis (Chilean wine palm); Jubaea spectabilis (syrup palm); Juglans sp.; Juglans spp.; Juniperus communis (common juniper); Juniperus oxicedrus (prickly juniper); Juniperus serayschanica? (Zerayschan juniper); Juniperus sp.; Juniperus spp.; Kalmia latifolia (mountain laurel); Karwinskia humboldtiana (Coyotillo); Klainedoxa gabonensis; Knightia excelsa (rewarewa); Krugiodendron ferreum; Kunzea baxteri (mountain bush); Laguncularia racemosa (white mangrove); Larix decidua (European larch); Lathyrus latifolius; Lavandula sp. (lavender); Ledum palustre (marsh tea); Leptoderris hypargyrea; Leptospermum laevigatum (Australian tea tree); Leptospermum scoparium (tea tree/rose gem); Leucadendron argenteum (silver tree); Leucaena retusa; Leucobryum glaucum; Leucospermum reflexum; Ligustrum lucidum (glossy privet); Liquidambar spp.; Liquidambar styraciflua (sweet gum); Liquidambar styraciflua (shade) (sweet gum); Liquidambar styraciflua (sun) (sweet gum); Liriodendron spp.; Liriodendron tuhpifera (tulip tree); Livistona chinensis (Chinese fountain palm); Lomatia fraxinifolia (ashleaf lomatia); Lophira alata; Lophira lanceolata; Lophira spp.; Lotus corniculatus var arvensis; Lotus pedunculatus; Luipus albicanlis; Maackia chinensis; Macadamia ternifolia (Queensland nut); Macaranga triloba (Macaranga); Maesopsis eminii; Magnolia grandiflora (magnolia); Magnolia spp.; Mahonia spp. (Oregon grape); Malaleuca leucadendron (paper bark); Malus domestica (apple); Malvaviscus arboreus; Mangifera indica (mango); Mattuccia struthiopteris (ostrich plume fern); Medicago sativa (alfalfa); Melaleuca linariifolia (bottlebush melaleuca); Melaleuca quinquenervia (cajeput tree); Melia spp.; Metasequoia spp. (metasequoie); Metrosideros excelsa (New Zealand Christmas tree); Metrosideros kermadecensis (irontree); Millettia sp.; Molinia caerulea (purple moor grass); Morus spp.; Mucuna deeringeniana (velvet bean); Mucuna pruriens var. utilis (velvet bean); Mucuna sp. (velvet bean); Myrianthus arboreus; Myrica califormica (Pacific bayberry); Myrica cerifera (southern bayberry); Myrica mexicana (Mexican wax-myrtle); Myrica spp.; Myrtus communis (true/common myrtle); Myrtus pedunculata (myrtle); Myrtus spp. (myrtle); Nandina domestica (heavenly/sacred bamboo); Nandina spp. (dwarf golden bamboo); Nauclea latifolia; Neckera pennata; Nerium oleander (oleander); Nyssa spp.; Ochna pulchra (ochnea); Olea sp. (olive); Olneya spp. (ironwood); Olneya tesota (desert ironwood); Opuntia lindheimeri; Oryza sativa (rice (M202)); Oxydendrum spp.; Pachyra insignis; Pancovia laurentii; Parinari cunatellifolia; Parrotia persica (Persian parrotia); Parrotiopsis jacquemontiana; Pellaea rotundifolia; Persea spp.; Phaseolus vulgaris (bean); Philonotis fontanum; Phoenix dactylifera (date palm); Phoenix reclinata; Phragmites mauritianum (reed); Picea abies (Norway spruce); Picea alcoquiana; Picea asperata (dragon spruce); Picea aurantiaca (yellowtwig spruce); Picea engelmanii (Engelman spruce); Picea glauca (white spruce); Picea koyamai (koyama spruce); Picea likiangensis var. (Balfour spruce); Picea mariana (black spruce); Picea montigena (candelabra spruce); Picea morrisonicola; Picea omorika (Serbian spruce); Picea orientalis (Oriental spruce); Picea pungens (Colorado blue spruce); Picea rubens (red spruce); Picea sitchensis (Sitka spruce); Picea smithiana (Himalayan spruce); Picea spp. (spruce); Pictetia aculeata; Pinus densiflora (red pine); Pinus halepensis (Aleppo pine); Pinus pinea (umberella/Italian stone pine); Pinus pinea (June) (umberella/Italian stone pine); Pinus pinea (May) (umberella/Italian stone pine); Pinus pinea (October) (umberella/Italian stone pine); Pinus ponderosa (June) (ponderosa pine); Pinus ponderosa (September) (ponderosa pine); Pinus sibirica (Siberian pine); Pinus sp.; Pinus spp.; Pinus sylvestris (Scots pine); Pinus taeda (loblolly pine); Pisonia albida; Pistacia lentiscus (pistachio); Pisum sativum (pea); Pittosporum tobira (Chinese pittosporum); Pittosporum undulatum (orange berry pittosporum); Platanus acerifolia (London planetree); Platanus hybrida; Platanus occidentalis (American sycamore); Platanus orientalis (Asian planetree); Platanus racemosa (California/western sycamore); Platanus sp. (plane); Platanus spp.; Pleioblastus distichus (fern bamboo); Pleurozium schreberi; Podocarpus gracilior (fern pine); Polygonum schalinense (Japanese knotweed); Polypodium lucidum; Polypodium punctatum; Polytrichum commune (deciduous/hair moss); Polytrichum juruperinum; Polytrichum piliferum; Populus alba (white poplar); Populus balsamifera (balsam poplar); Populus balsamifera ssp trichocarpa (black cottonwood); Populus deltoides (eastern poplar/cottonwood); Populus fremontii (Fremont cottonwood); Populus grandidentata (bigtooth aspen); Populus koreana; Populus maximowiczii; Populus nigra (black poplar); Populus sp.; Populus spp.; Populus szechuanica (Szechuan poplar); Populus tremula; Populus tremuloides (quaking aspen); Populus trichocarpa (western balsam poplar); Protea obtusifolia (bluntleaf protea); Prunus sp.; Prunus spp.; Pseudospondias microcarpa; Pseudotsuga menziesii ssp menziessi (coastal douglas fir); Pseudotsuga spp.; Pteridium aquilinum (bracken); Pterocarpus luscens; Pterocarpus soyauxii; Ptilium crista-castrensis; Pueraria javanica; Pueraria lobata (kudzu bean); Pueraria spp.; Pyrus kawakamii (evergreen pear); Pyrus malus (See Malus pumila); Quercus agrifolia (Californian live oak); Quercus alba (American white oak); Quercus bicolor (swamp white oak); Quercus borealis (rubra) (red oak); Quercus bushii (bush oak); Quercus calliprinos (Palestina oak); Quercus canariensis (canary oak); Quercus cerris (Turkey oak); Quercus chrysolepis (canyon live oak); Quercus coccifera (kermes oak/grain tree); Quercus coccinea (scarlet oak); Quercus douglasii (blue oak); Quercus dumosa (California scrub oak); Quercus durata (leather oak); Quercus engelmanii (Engelmann oak); Quercus faginea (Portuguese oak); Quercus falcata (southern red oak); Quercus frainetto (Hungarian oak); Quercus gambelii (gambel oak); Quercus garryana (Oregon white oak); Quercus glauca (Japanese blue oak); Quercus ilex (holm/holly/evergreen oak); Quercus ilex (June) (holm/holly/evergreen oak); Quercus ilex (May) (holm/holly/evergreen oak); Quercus ilex (October) (holm/holly/evergreen oak); Quercus ilex (sun-exposed) (holm/holly/evergreen oak); Quercus imbricaria (shingle oak); Quercus incana (bluejack oak); Quercus ithaburiensis; Quercus kelloggia (California black oak); Quercus laevis (scrub/Turkey oak); Quercus laurifolia (laurel oak); Quercus libani (Lebanon oak); Quercus lobata (California white/valley oak); Quercus macrocarpa (bur oak); Quercus macrolepis (valonia oak); Quercus mexicana (Mexican oak); Quercus morehus (oracle oak); Quercus myrtiflora (myrtle oak); Quercus nigra (water oak); Quercus palmeri (Palmer oak); Quercus palustris (pin oak); Quercus petraea (sessile/durmast oak); Quercus phellos (willow oak); Quercus phillyraeoides; Quercus prinus (swamp chestnut oak); Quercus pubescens (pubescent oak); Quercus pyrenaica (Pyrenees oak); Quercus r. var. concordia (golden oak); Quercus robur (pedunculata) (English/European/pendunculate oak); Quercus rotundifolia; Quercus rubra (See borealis); Quercus rudkinii (Rudkin oak); Quercus serrata (acutissima) (live oak); Quercus spp.; Quercus stellata (post oak); Quercus suber (cork oak); Quercus trojana (Macedonian oak); Quercus variabilis (Oriental oak); Quercus velutina (black oak); Quercus virginiana (Virginia live oak); Quercus wislizenii (interior live oak); Raphia farinifera; Reynosia guama; Reynosia mangle; Rhamnus alaternus (Italian buckthorn); Rhamnus califormica (California buckthorn); Rhamnus crocea/ceanothus (redberry); Rhamnus lycioides (June); Rhamnus lycioides (September); Rhamnus purshiana (cascara buckthorn); Rhamnus Rhaphiolepis indica (India hawthorne); Rhapis humilis (slender lady palm); Rhizophora mangle (red mangrove); Rhus leptodictya (sumac); Rhus ovata (Sugarbush/sugar sumac); Rinorea sp.; Robinia pseudoacacia (black locust); Robinia spp. (locust); Romneya coulteri (matilija poppy); Romneya trichocalyx (bristlecup matilija poppy); Rosmarinus officinalis (rosemary); Roystonea elata (royal palm); Rubus spp.; Rubus ursinus (blackberry); Sabal palmetto (cabbage palmetto); Salix alba (white willow); Salix amygdaloides (peach leaf willow); Salix atrocineria; Salix babylonica (weeping willow); Salix caroliniana (coast plain willow); Salix discolor (pussy willow); Salix hindsiana (sandbar willow); Salix interior (sandbar willow); Salix lasiandra (Pacific willow); Salix lasiolepis (arroyo willow); Salix lutea (yellow willow); Salix matsudana (corkscrew willow); Salix nigra (black willow); Salix pentandra (bay leaved willow); Salix phylicifolia (tea leafed willow); Salix scouleriana (scouler willow); Salix sp.; Salix spp.; Salvia sp. (sage); Sasa palmata (sasa bamboo); Sclerocarya birrea (S. caphra) (Maroala plum); Secale cereale (rye); Securinega virosa (securinega); Serenoa repens (saw palmetto); Simmondsia chinensis (Arizona jojoba); Sinobambusa (tootsik); Sophora japonica (Japanese pagoda tree); Sorbus aucuparia (mountain ash); Sorbus scopulina (mountain ash); Sorghum sp. (sorghum); Spartium junceum (Spanish broom); Spartium junceum (May) (Spanish broom); Spartium junceum (October) (Spanish broom); Sphagnum capillifolium; Sphagnum fuscum; Sphagnum girgensohnii; Stenocarpus sinatus (firewheel tree); Stiolobium deerangianum (velvet bean); Symphoricarpus occidentalis (snowberry); Syncarpia glomulifera (turpentine tree); Syzygium guineense; Taxodium spp. (cypress); Tecomaria capensis (Cape Honeysuckle); Tectaria cicutaria; Templetonia retusa (red coral bush); Terminalia prunoides (myrobolan family); Terminalia sericea (myrobolan family); Terminalia superba; Thelypteris decursive-pinnata (maiden fern); Thelypteris dentata; Thelypteris kunthii; Thelypteris plegopteri (beech fern); Thelypteris spp.; Thrinax morrisii; Thuja occidentalis (northern white cedar); Thuja orientalis (Chinese arbor vitae); Thuja plicate (western red cedar); Thuja spp.; Tilia americana (American linden); Trachycarpus fortunei (windmill palm); Trifolium pratense (red clover); Trilepisium madagascariense; Tristania conferta (Brisbane box); Tristania laurina; Triticum aestivum (wheat); Tsuga heterophylla (western hemlock); Tsuga mertensiana (coastal hemlock); Tsuga spp.; Uapaca heudelotii; Ulex europaeus (adult) (gorse); Ulex europaeus (young) (gorse); Ulex parviflorus (June) (gorse); Ulex parviflorus (September) (gorse); Ulmus americana (American elm); Ulmus parviflora (Chinese elm); Ulmus spp. (elm); Vaccinium myrtillus (bilberry); Vaccinium spp.; Vaccinium uliginosum (Waccinium?) (blueberry); Vaccinium vitis-ideae (cowberry/red-bilberry); Vigna unguiculata; Vinca minor (periwinkle); Vitis vinifera (grape (chardonnay)); Vitis vinifera (grape (chardonnay) with fruit); Washingtonia filifera (California fan palm); Washingtonia robusta (Mexican fan palm); Washingtonia spp.; Wisteria floribunda (Japanese wisteria); Wisteria spp. (wisteria); Xanthocephalum dracuna (perennial broomweed); Xylosma congestum (shiny xylosma); Zanthoxylum fagara (Shinner's sickle tongue); Zea mays (corn); or Ziziphus obtusifolia.

Mosses (Bryophytes) that Naturally Produce Isoprene

In another aspect, provided here is subject matter that harnesses organisms that naturally produce volatile organic molecules, including for example the organic molecule isoprene. In some instances, the organism is a Bryophyte and the Bryophyte naturally releases isoprene without genetic modification of the organism. Exemplary Bryophytes for use with the device include, but are not limited to: Atrichum undulatum, Brotherella recurvans, Dicranum polysetum, Hylocomium splendens, Leucobryum glaucum, Neckera pennata, Philonotis Fontana, Pleurozium schreberi, Polytrichum commune, Polytrichum juniperinum, Polytrichum piliferum, Ptilium crista-castrensis, Sphagnum capillifolium, Sphagnum fuscum, Sphagnum girgensohnii, or Aulacomnium palustre. In some instances, the Bryophyte is Polytrichum juniperinium. In some instances, the Bryophyte is Atrichum undulatum.

### Genetically Modified Mosses (Bryophytes)

Another aspect of the subject matter described herein is an organism that has been genetically modified to produce increased levels of a volatile organic molecule of interest, including the modification of organisms that do not naturally produce a particular volatile organic molecule of interest. In some embodiments, provided herein is the modification of an organism to produce increased levels of a particular volatile organic molecule of interest.

In some instances, an organism that is part of the devices provided herein has been genetically modified to produce increased levels of isoprene compared to the same organism that has not been genetically modified. Provided herein are specific examples of genetic modification of certain Bryophyte species. However, the present disclosure contemplates genetic transformation of all organisms provided herein. For example, in some embodiments, provided herein is the genetic modification of a suitable member of the plant kingdom using Agrobacterium tumefaciens to insert a plasmid into the plant. In some instances, the organism has been genetically modified to express a native or modified form of the isoprene synthase enzyme. In some instances, the organism has been genetically modified to generate a terpene of organic molecule product. In some instances, the organism has been genetically modified to reduce glucose biosynthesis. In some instances, the organism has been genetically modified to reduce terpenoid biosynthesis.

In some instances, the organism has been genetically modified to reduce the activity of the glucose-6-phosphate isomerase enzyme or to reduce the activity of PEP (phosphoenolpyruvate) carboxylase enzyme. The PEP carboxylase enzyme becomes activated at increased CO₂ levels and can reduce yields of isoprene in natural systems.

In some instances, the organism has been genetically modified to reduce the activity of the dimethylallyl trans transferase enzyme or the farnesyl trans transferase enzyme, or both. In some instances, the organism has been genetically modified to increase the activity of an enzyme of the DOXP pathway.

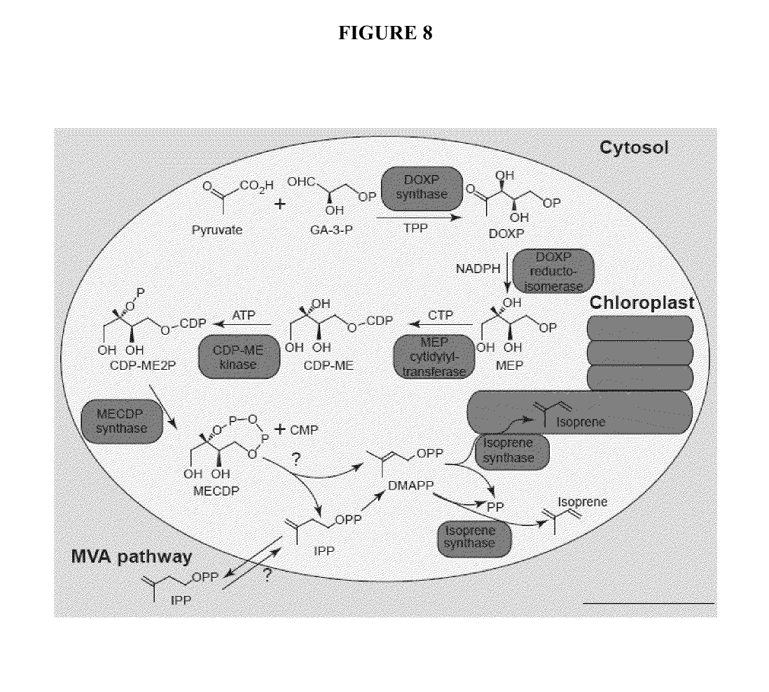

FIG. 8 is a schematic depiction of the DOXP biosynthetic pathway that leads to isoprene formation in plants. Abbreviations in FIG. 8 include: IPP, isopentenyl pyrophosphate; DMAPP, dimethylallyl pyrophosphate; CDP-ME, 4-(cytidine 59-diphospho)-2-C-methyl-D-erythritol; CDPME2P, 2-phospho-4-(cytidine 59-diphospho)-2-C-methyl-D-erythritol; DMAPP, dimethylallyl diphosphate; DOXP, 1-deoxy-D-xylulose-5-phosphate; GA-3-P, glyceraldehyde-3-phosphate; IPP, isopentenyl pyrophosphate; MECDP, 2-C-methyl-D-erythritol 2,4-cyclodiphosphate; MEP, 2-C-methyl-D-erythritol-4-phosphate; MVA, mevalonic acid. Glyceraldehyde-3-phosphate (GAP) is formed during the Calvin cycle of carbon dioxide fixation through ribulose bisphosphate carboxylase (RuBisCo), while pyruvate is formed through from GAP through the lower half of the glycolytic pathway.

In some instances, the genetically modified Bryophyte is Physcomitrella patens. In some instances, the genetically modified Bryophyte is Ceratodon purpureus. In an embodiment, the transformation of Ceratodon purpureus is carried out with a similar approach to the example of modifying Physcomitrella patens as described herein.

### Members of the Fungi Kingdom

In another aspect, provided herein is a biological solar panel device comprising a photosynthetic or photosynthetic-like organism that is a member of the Fungi kingdom. In some embodiments, the organism is a member of the Ascomycota division. In some embodiments, the organism is a member of the Acarosporales order. In some embodiments, the organism is a member of the Acarosporaceae family. In some embodiments, the organism is a member of the Agyriales division. In some embodiments, the organism is a member of the Agyriaceae or Anamylosporaceae family. In some embodiments, the organism is a member of the Arthoniales order. In some embodiments, the organism is a member of the Arthoniaceae, Chrysothricaceae, Melaspileaceaem or Roccellaceae family. In some embodiments, the organism is a member of the Lecanorales order. In some embodiments, the organism is a member of the Catillariaceae, Cladoniaceae, Lecanoraceae, Parmeliaceae, Ramalinaceae, or Stereocaulaceae family. In some embodiments, the organism is a member of the Lichinales order. In some embodiments, the organism is a member of the Gloeoheppiaceae, Heppiaceae, Lichinaceae, or Peltulaceae family. In some embodiments, the organism is a member of the Ostropales order. In some embodiments, the organism is a member of the Gomphillacaeae, Graphidaceae, Gyalectaceae, Stictidaceae, or Thelotremataceae family. In some embodiments, the organism is a member of the Peltigerales order. In some embodiments, the organism is a member of the Collemataceae, Lobariaceae, Nephromataceae, Pannariaceae, Peltigeraceae, or Placynthiaceae family. In some embodiments, the organism is a member of the Pertusariales order. In some embodiments, the organism is a member of the Megasporaceae or Pertusariaceae family. In some embodiments, the organism is a member of the Pyrenulales order. In some embodiments, the organism is a member of the Monoblastiaceae or Pyrenulaceae family. In some embodiments, the organism is a member of the Teloschistales order. In some embodiments, the organism is a member of the Letroutiaceae, Physciaceae, or Teloschistaceae family. In some embodiments, the organism is a member of Verrucariales order. In some embodiments, the organism is a member of the Verrucariaceae family. In some embodiments, the organism is a member of Incertae sedis order. In some embodiments, the organism is a member of the Arthrorhaphidaceae (Ostropomycetidae), Arthopyreniaceae (Dothideomycetes), Elixiaceae (Lecanoromycetes), Microtheliopsidaceae (Dothideomycetes), Pyrenotrichaceae (Dothideomycetes), Lecideaceae (Lecanoromycetidae), or Trypetheliaceae (Dothideomycetes) family. In some embodiments, the organism is a member of the Basidiomycota division.

### Members of the Protista Kingdom

Another aspect of the subject matter described herein is a biological solar panel device comprising a photosynthetic organism and the photosynthetic organism is a member of the Protista Kingdom. In particular embodiments, the protists provided herein are photosynthetic. In further or additional embodiments, the photosynthetic protist is an algal protist. Photosynthetic algal protists have a nutrition that is plant-like. In some embodiments, the protist is a member of the Dinoflagellata division.

In some embodiments, the protist is a member of Dinoflagellata division and is plankton, including “red” planton that are responsible for phenomena referred to as algal bloom and “Red Tide.”

In some embodiments, the protist is a member of the Euglenophyta. For example, in some embodiments the protist is a Euglena. Euglenas have a flagellum, chloroplast, and photosynthesize when subjected to light.

In additional embodiments, the protist is a member of the Chlorophyta division. For example, in some embodiments the protist is a green algae. The chloroplasts of green algae are similar to plants (containing chlorophyll B). Various species of green algae may be found in a variety of environments including both fresh and salt water, damp soil, the surface of snow, and within other organisms (lichens, hydra, polar bear hair), as described herein. In some embodiments, the protist is a chlamydomonas, a volvox, an ulva, a closterium, or a spirogyra.

In further embodiments, the protist is a member of the Phaeophyta division. For example, in the some embodiments, the protist is a brown algae. In some embodiments, the protist is a member of the genera Fucus and/or Laminaria.

In still further embodiments, the protist is a member of the Rhodophyta division. In some embodiments, the protist is a red algae.

### Members of the Monera Kingdom

In some embodiments, the photosynthetic or photosynthetic-like organism is living and is a member of the traditional Monerans kingdom (e.g., including eubacteria and archaebacteria). In some embodiments, the organism is a member of the Cyanophycota phylum. In some embodiments, the organism is a member of the Cyanophyceae class. In some embodiments, the organism is a member of the Chroococcales order. In some embodiments, the organism is a member of the Chroococcaceae family. In some embodiments, the organism is a member of the Synechocystis genus.

### Symbionts

In a further aspect of the subject matter provided herein, provided is a symbiont pair, trimer, or other combination of plural organisms. Organisms that are symbiotic are genetically distinct organisms that utilize each other in a beneficial way for each organism.

In some embodiments, the organism is a symbiont, including for example a lichen-forming species. In further or additional embodiments, provided herein is a lichen. In certain embodiments, the lichen is within a class of isoprene emitting organisms. In some embodiments, the lichen is a symbiont of fungi and cyanobacteria. In some embodiments, the devices provided herein comprise a symbiont to immobilize and hydrate a fungus or cyanobacterium, whereby the volatiles are then collected from the device.

In some embodiments, the organism is a symbiont of a fungus or algae species. In some embodiments, the symbiont is a lichen. In some embodiments, the lichen provided herein immobilizes or hydrates an algae and/or cyanobacterium.

### Nitrogen-Fixing Bacteria

In a further aspect, provided herein are biological solar panel devices that comprise a nitrogen fixing bacterium. In some embodiments, the device comprises an organism that further comprises a nitrogen-fixing bacterium. In some embodiments, the nitrogen-fixing bacteria is Azospirillum braziliense.

Gas Layer

In another aspect, provided herein is a biological solar panel device comprising a gas layer. For example, as illustrated in FIG. 5, in certain situations the gas layer is an air gap. In some embodiments, a gas layer of the devices provided herein comprises carbon dioxide. In some embodiments, the gas layer comprises at least one volatile organic molecule that has been released from a photosynthetic organism. In some embodiments, the gas layer is pressurized using compressed feed air. In some embodiments, the gas layer comprises air or is mixed with atmospheric air. In some instances, the gas layer comprises a gas that increases the growth the photosynthetic layer. In some instances, the gas layer comprises a gas that inhibits the growth the photosynthetic layer. In some instances, the gas layer has a certain flow rate through the device. In some instances, the gas layer comprises gas that travels through a permeable transparent or translucent material. In some instances, the gas layer comprises gas does not travel through a transparent or translucent material. In some instances, the gas layer comprises nitrogen. In some instances, the gas layer comprises oxygen.

Support Layer

In another aspect, provided herein is a biological solar panel device comprising a support layer. For example, as illustrated in FIG. 5, in certain situations the support layer is a vegetation support layer. In some embodiments, a support layer of a device herein is a vegetation support layer. In some embodiments, the support layer comprises soil. In some embodiments, the support layer comprises water.

In some embodiments, the support layer further comprises a feed layer.

In some embodiments, the support layer is porous to nutrients.

In some embodiments, the photosynthetic layer is integrated into the support layer.

In some embodiments, the support layer is a mesh. In some embodiments, the support layer is substantially impermeable to the volatile organic molecule passing through the support layer. In some embodiments, the support layer is removable from the device.

In some embodiments, the support layer permits the passage of water into the support layer without allowing for the release of a volatile organic molecule from the device. In some embodiments, the support layer immobilizes the photosynthetic layer. In some embodiments, the support layer provides dimensional support to the photosynthetic layer. In some embodiments, the support layer further comprises filter paper.

In some embodiments, the support layer prevents escape of the product through the bottom of the device, and holds organisms in place. In some embodiments, the layer allows for wicking of water from below the device without allowing substantial isoprene to pass through. In some embodiments, the support layer comprises a fabric.

Biological Solar Panel Array

In some embodiments, more than one of any of the devices described herein are connected to form an array. In some embodiments, an array comprises at least ten biological solar panel devices or at least twenty five biological solar panel devices. In some instances, an array is arranged in a grid. In some instances, the array is in communication with one or more supply systems. In some instances, the array is in communication with one or more collection systems.

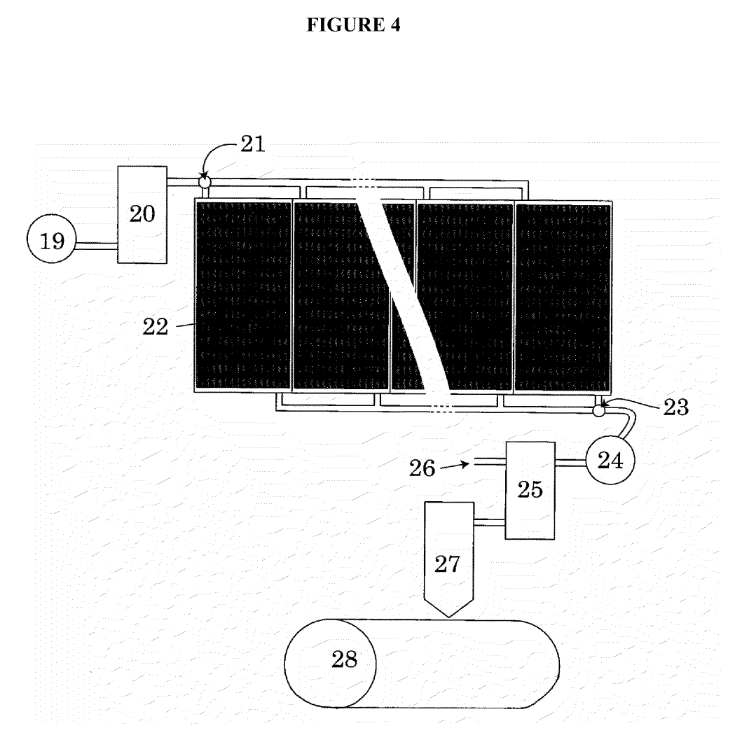

FIG. 4 demonstrates an exemplary and non-limiting embodiment of an example of a biological solar panel array with collection apparatus. As illustrated in FIG. 4, after creation (e.g., synthesis) of a volatile product (isoprene, for example) is complete, purge air enters the system through an inlet 19. Air passes through the filter/humidifier/heater 20 and into the solar panel array 22 through a valve 21. In certain situations, air enriched in the synthesis product exits the array 22 through valve 23. The effluent is compressed by pump 24, and enters the hollow fiber unit 25, which contains a hydrophobic canister that allows organic molecules to pass through while water vapor is retained. In certain embodiments, waste gas is vented through vent 26 while pass-through gas enriched in product and depleted in water vapor is condensed in condenser 27 and collected in tank 28.

Another aspect of the subject matter provided herein is the placement of the biological solar panel devices and arrays in certain areas to maximize the production and collection of a organic molecule. For example, in some embodiments, a device, or an array of devices, is placed in a desert. Placement in the desert provides for an excellent source of natural sunlight to a photosynthetic organism. In still further embodiments, the devices and arrays provided herein are placed in a structure of a building (e.g., a roof, wall, side, and the like). In still further embodiments, the device or array of devices is incorporated into a developed structure (e.g., a building). In still further embodiments, the devices and arrays provided herein are manufactured to be provided in any suitable environment (including fore example in-doors or outside).

### Terpenes and Organic Molecules

In another aspect of the subject matter provided herein, a material or organism of a biological solar panel device produces a volatile organic molecule that comprises at least one terpene.

Terpenes are a large and varied class of organic molecules, produced primarily by a wide variety of photosynthetic organisms. When terpenes are modified chemically, such as by oxidation or rearrangement of the carbon skeleton, the resulting compounds are generally referred to as terpenoids or isoprenoids. Terpenoids or isoprenoids generally comprise a heteroatom. Terpenes can be the primary constituents of the essential oils of many types of plants and flowers. Essential oils are used widely as natural flavor additives for food, as fragrances in perfumery, and in traditional and alternative medicines, such as aromatherapy. Synthetic variations and derivatives of natural terpenes also greatly expand the variety of aromas used in perfumery and flavors used in food additives.

Terpenes are a diverse class of biosynthetic organic molecules comprising multiple units of isoprene (2-methyl-buta-1,3-diene), which is a five-carbon organic molecule. Isoprene has the following chemical structure:

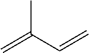

Isoprene reportedly has a density of 0.681 g/cm³, a melting point of about −146° C. and a boiling point of about 34° C.

Isoprene units can be linked together to form acyclic (including branched or linearly arranged carbon atoms) or cyclic framework. The size of an isoprenoid refers to the total number of the carbon atoms of the isoprenoid framework, and is typically a multiplicity of five.

In some embodiments, a volatile organic molecule of the devices provided herein comprises at least one terpene or at least one terpenoid, or a combination of at least one terpene and at least terpenoid. As described herein, terpenes the terpenoids are classified according to the number of isoprene units, for example:

Hemiterpenes and Hemiterpenoids contain 1 isoprene unit

Monoterpenes and Monoterpenoids contain 2 isoprene units

Sesquiterpenes and sesquiterpenoids contain 3 isoprene units

Diterpenes and Diterpenoids contain 4 isoprene units

Sesterterpenes and Sesterterpenoids contain 5 isoprene units

Triterpenes and Triterpenoids contain 6 isoprene units

Tetraterpenes and Tetraterpenoids contain 8 isoprene units

Polyterpenes and Polyterpenoids contain more than 8 ispoprene units

### Terpenes

In some embodiments, the terpene is a hemiterpene. In some embodiments, the hemiterpene is isoprene, cyclopentene, or piperylene. In some embodiments, the hemiterpene is isoprene. Isoprene are reactive molecule that can be used in the production of synthetic rubber, or to generate a variety of combustible fuel molecules, via known chemical processes such as hydrogenation, a catalyzed Diels Alder or metathesis reaction pathway (for example, according to U.S. Pat. Publ. No. 20090043144), or cationic oligomerization to form di- and triterpenes (for example using the monoterpene α-pinene to form a diterpene and/or triterpene). In particular, these approaches will lead to 5-15 carbon organic molecules, within the range of present gasoline composition (comprised largely of four to twelve carbon organic molecule molecules), with approximately equivalent energy content (since none of the molecules is oxygenated). Subsequent processing of isoprene can remove reactive double bond(s) from the molecule to improve stability and reduce volatility, so that the product(s) may be used as a liquid fuel.

In some embodiments, in the terpene is a monoterpene. In some embodiments, the monoterpene is limonene, 2,6-dimethyloctane, α-myrcene, β-myrcene, ocimene, pinane, camphane, isocamphane, fenchane, carene, cymene, camphene, sabinene, tricyclene, fenchene, β-fenchene, limonene, β-phellandrene, β-phellandrene, α-pinene, β-pinene, sabinene, α-terpinene, α-terpinene, β-terpinene, α-thujene, and β-thujene. As one example, α-pinene, a major component of turpentine, is obtained as provided herein and used in internal combustion engines.

In some embodiments, the terpene is a sesquiterpene. In some embodiments, the sesquiterpene is farnesane, farnesene, bisabolane, bisabolene, zingiberene, cyclofarnesane, farnesene, sesquiphellandrene, sesquisabinene, sesquithujene, sesquicarene, germacrane, germacrene, bicycloelemene, humulane, humulene, eudesmene, eudesmane, eremophilane, eremophilatriene, ishwarane, valerane, guajane, guajazulene, or longifolene.

In some embodiments, the terpene is a diterpene. In some embodiments, the diterpene is cafestol, kahweol, cembrene, or taxadiene.

In some embodiments, the terpene is a sesterterpene. In some embodiments, the sesterterpene is geranylfarnesol.

In some embodiments, the terpene is a triterpene. In some embodiments, the triterpene is squalene.

In some embodiments, the terpene is a tetraterpene. In some embodiments, the tetraterpene is lycopene or carotene.

In some embodiments, the terpene is a polyterpene. In some embodiments, the polyterpene is gutta-percha.

### Terpenoids

In some embodiments, a device herein comprises a volatile organic molecule that comprises at least one terpenoid.

In some embodiments, the terpenoid is a hemiterpenoid. In some embodiments, the hemiterpenoid is an oxygen-containing derivative of isoprene. In some embodiments, the oxygen-containing derivative of isoprene is prenol (methyl butenol), isovaleric acid, (S)-3-methyl-3-buten-2-ol, tiglic acid, angelic acid, or 3-methylbutenoic acid.

In some embodiments, the terpenoid is a monoterpenoid. In some embodiments, the monoterpenoid is 1,8-cineole, borneol, camphor, carvacrol, citral, citronellal, citronellol, eucalyptol, geraniol, lavandulol, linalool, menthol, isomenthol, neomenthol, neoisomenthol, pulegol, isopulegol, piperitol, α-terpenol, carveol, perillaldehyde, menthone, isomenthone, pulegone, isopulegone, phellandral, piperitone, dihydrocarvone, carvenone, carvone, nerol, neral, (S)-(+)-dihydrotagetone, (E)-tagetone, thymol, cineol, eucalyptol, ascaridol, cuminaldehyde, myrcenol, ipsdienol, (2R,4R)-trans-rose oxide, (2S,4R)-cis-rose oxide, (+)-trans-chrysanthemic acid, (+)-trans-pyrethric acid, (+)-chrysanthemol, (+)-cinerolone, (+)-jasmolone, (+)-pyrethrolone, cinerin, jasmolin, pyrethrin, junionone, (1S,2S)-fragranol, or (1R,2S)-grandisol.

In some embodiments, the terpenoid is a sesquiterpenoid. In some embodiments, the sesquiterpenoid is lactaroviolin, furanoeremophilane, valeranone, eudesmol, costus acid, costol, santonan, taurin, santonin, tubipofuran, furanoeudesmadiene, furanoeudesmadienone, bisabolol, dendrolasin, sesquirosefuran, longifolin, sinsenal, abscisic acid, artemisinins, cadinene, caryophyllene, copaene, farnesol, gossypol, shyobunol, germacrone, elemenone, guaiane, guaiazulene, humulene, patchoulol, santonin, or a trichothecene.

In some embodiments, the terpenoid is a diterpenoid. In some embodiments, the diterpenoid is andrographolide.

In some embodiments, terpenoid is a sesterterpenoid. In some embodiments, the sesterterpenoid is manoalide.

In some embodiments, the terpenoid is a triterpenoid. In some embodiments, the triterpenoid is a phytosterol.

In some embodiments, the terpenoid is a tetraterpenoid. In some embodiments, the tetraterpenoid is trianthenol.

In some embodiments, the terpenoid is a polyterpenoid.

Other Volatile Compounds

In some embodiments, a device herein comprises a volatile organic molecule that is an essential fatty acid, a benzenoid, an aldehyde, or a heterocycle. In some embodiments, the essential fatty acid is a short chain polyunsaturated fatty acid or a long chain polyunsaturated fatty acid. In some embodiments, the short chain polyunsaturated fatty acid is a ω-3 fatty acids (e.g., α-linolenic acid (ALA (18:3)) or a ω-6 fatty acids (e.g., linoleic acid (LA (18:2))). In some embodiments, the long chain polyunsaturated fatty acid is a ω-3 fatty acids (e.g., eicosapentaenoic acid (EPA (20:5)), docosahexaenoic acid (DHA (22:6))), or a ω-6 fatty acids (e.g., gamma-linolenic acid (GLA (18:3)), dihomo-gamma-linolenic acid (DGLA (20:3)), or arachidonic acid (AA (20:4))). In some embodiments, the volatile organic molecule is a benzenoid. In some embodiments, the benzenoid is salicylate or benzoate. In some embodiments, the volatile organic molecule is an aldehyde. In some embodiments, the aldehyde is hexanal or nonenal. In some embodiments, the heterocycle is bicyclic. In some embodiments, the bicyclic heterocycle is indole.

### Methods of Manufacture

A method of manufacturing a device herein is provided. In some embodiments, the device is manufactured through processes similar to those that are presently used to make various planar assemblies, such as transplantable turf (for example U.S. Pat. No. 5,189,833), thermopane (gas filled) multilayer windows, sheet plastic, or carpeting. In some embodiments, the photosynthetic layer is generated in situ (through photosynthetically-supported growth on the ‘vegetation support layer’), or coated onto a planar surface. In some embodiments, the photosynthetic layer is immobilized, such that it remains dimensionally intact upon subsequent manipulation. In some embodiments, the air gap need not be rigid (but can be), and may optionally is generated from positive pressure inside the assembly. In some embodiments, a device comprising multiple layers of photosynthetic organisms are manufactured as provided herein. In some embodiments, the support layer is hydroponic. In some embodiments, the support layer is soil-based and is porous to nutrients (for example, a fabric or paper). In some embodiments, the support layer doesn't absorb the volatile organic molecule product or allow it to escape.

### Methods and Systems for the Collection of Organic Molecules

In another aspect, provided herein is a method comprising collecting isoprene that has been released from a living or non-living material that is in a photosynthetic layer of a biological solar panel device.

In some embodiments of the methods, systems and devices provided herein, provided is a feed gas has been introduced into an inlet of the biological solar panel array. In some embodiments, a volatile organic molecule has been withdrawn from an outlet of a biological solar panel array. In some embodiments, the feed gas at the inlet comprises carbon dioxide. In some embodiments, the feed gas is continuously introduced into the inlet. In some embodiments, the feed gas is periodically introduced into the inlet.

FIG. 4 illustrates an exemplary and non-limiting method of collection of a product from a device herein, for example the condenser 27 and collection tank 28 features.

### Pressure Swing Adsorption

In some embodiments of the methods and systems described herein, isoprene as a volatile organic molecule is collected using a pressure swing adsorption (PSA) unit that separates the isoprene from other components in the gas layer. In some embodiments, the volatile organic molecule is isoprene and the isoprene is collected using a vacuum pressure swing adsorption (VPSA) unit that separates other components in the gas layer from isoprene. In some embodiments, the feed gas is introduced into the inlet using a compressor. In some embodiments, the unit comprises an adsorbent material that is selective for binding components of the gas layer at a greater affinity than isoprene. In some embodiments, the unit selectively adsorbs isoprene using a diene and a catalyst. In some embodiments, the catalyst is platinum or charcoal. In some embodiments, the components comprise oxygen, carbon dioxide, water, argon, nitrogen, carbon monoxide, ozone, or oxides of nitrogen.

In some embodiments, the adsorbent material comprises a lithium exchanged zeolite. In some embodiments, the adsorbent material comprises monothabilamine, silica gel, activated carbon or alumina. In some embodiments, the unit comprises a pre-treatment layer that removes oxygen, carbon dioxide, and water. In some embodiments, a method includes providing a drive motor that drives the compressor. In some embodiments, a method includes providing a rechargeable power supply for the drive motor.

### Use of Solvents

In some embodiments of the methods and systems described herein, the volatile organic molecule is isoprene and the isoprene is collected by mixing together (a) a fluid containing isoprene that has been released from the photosynthetic organism and subsequently condensed and (b) a suitable organic molecule solvent that has a boiling point that is different than isoprene. Typically, a low boiling-point solvent is used. The solvent can be recycled (for example, via distillation and condensation) when the extract is concentrated. Exemplary solvents include, but are not limited to, hexane, carbon disulfide, petroleum ether, acetone and mixtures thereof.

In some embodiments, the solvent has a boiling point that is substantially different than the volatile organic molecule to be collected. For example, the boiling point of isoprene is about 34° C. Accordingly, in some embodiments, the boiling point of the solvent is different, in some cases substantially different, than about 34° C. (e.g., less than about 30° C. or greater than about 40° C.). In some embodiments, the volatile organic molecule has been condensed by transporting the volatile organic molecule underground for condensation.

### Polymerization

In some embodiments of the methods and systems provided herein, the collecting step comprises polymerizing the volatile organic molecule. In some embodiments, the volatile organic molecule is polymerized by cationic, anionic, free radical, or catalysis (e.g., Ziegler-Natta). Varying the conditions for polymerization can result in many different forms of matter, from solid (rubber) to low viscosity liquid forms, depending on the length of the polymer chains, their stereochemistry, crosslinking, etc.

In some embodiments, the volatile organic molecule is polymerized by catalysis (e.g., Ziegler-Natta).

### Hollow Fiber Gas Concentration

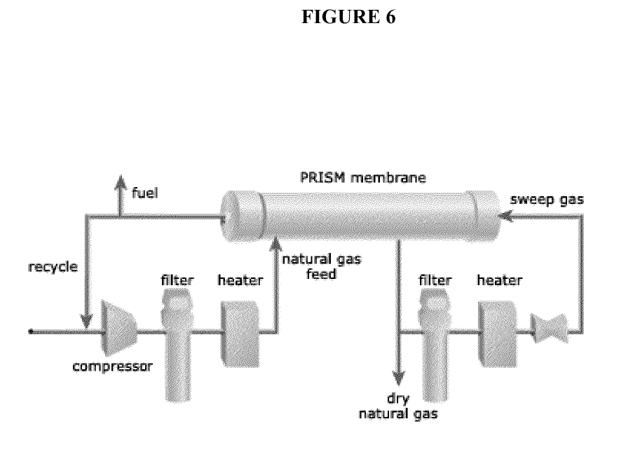

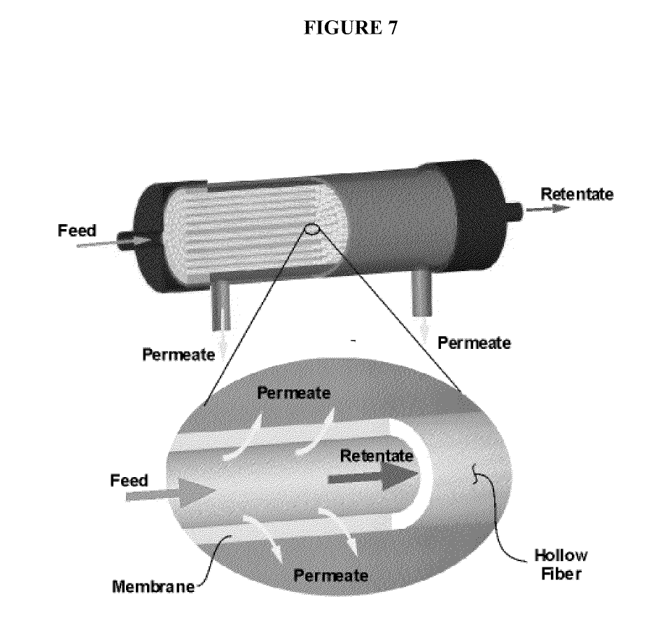

Several of the large companies in petrochemicals use hollow fiber gas concentration for gas separations. Air Products sells the Prism system. IGS (Generon) makes both PSA and membrane devices, and CUNO (now part of 3M). The system and method involves coating a supporting layer with a non-polar stationary phase. This phase will preferentially absorb organic molecules, and if the reverse phase material lies on a pressure gradient that separates the feed gas from a collection stream, then the organic molecule will be enriched on the collection side. In some embodiments, the collecting step comprises use of hollow fiber gas concentration. See, e.g., FIGS. 6 and 7.

### Use of a Capture Molecule

In some embodiments, the collecting step comprises use of a capture molecule. In some embodiments, the capture molecule is sulfur dioxide. An exemplary approach to capturing a volatile organic molecule is to react the organic molecule with sulfur dioxide in boiling methanol to generate a solid (for example, the five-membered cycloaddition sulfoxide adduct, R. L. Frank and R. P. Seven, in E. C. Horning, ed., Organic Syntheses, Collective Vol. III, John Wiley & Sons, Inc., New York, 1955, p. 499.; 46. R. L. Frank, C. E. Adams, J. B. Blegen, R. Deanin, and P. V. Smith, Ind. Eng. Chem. 39, 887 (1947).) The adduct of the organic molecule and capture molecule in this example melts at about 60 degrees, and can be recrystallized. Thus, the sulfoxide decomposes upon heating the solid to the organic molecule and SO₂.

### Induction of Isoprene Synthesis

In an aspect provided herein, a method comprises inducing isoprene synthesis of a material that uses carbon dioxide and water in the presence of sunlight to release a volatile organic molecule in a photosynthetic layer of a biological solar panel device wherein the biological solar panel device that comprises: (a) a layer comprising a material is transparent or translucent to light; (b) the photosynthetic layer comprising the material that uses carbon dioxide and water in the presence of sunlight to release a volatile organic molecule, wherein the photosynthetic layer is separated from the transparent or translucent material by a gas layer; and (c) a layer that provides support for the material that releases a volatile organic molecule.

In some embodiments, the temperature of the support layer that provides support for immobilizing a photosynthetic organism in the photosynthetic layer is maintained between about 25 to about 45° C. In some embodiments, isoprene synthesis of the photosynthetic organism is increased by introducing an organic compound or gas into the support layer. In some embodiments, the organic compound or gas is ethylene, nitric oxide or auxin.

In some embodiments, the photosynthetic organism is genetically modified such that isoprene synthase is placed under the control of a heat shock protein promoter.

Many plant genes are induced using light, possibly the best understood one uses a cryptochrome-activated response element CIB1, see Liu et al., Science 322 (5907)-1535-1539. In some embodiments, isoprene synthesis of the photosynthetic organism is increased by photoregulation.

In some embodiments, isoprene synthesis of the photosynthetic organism is increased by depriving the photosynthetic organism of a nutrient. In some embodiments, the nutrient is water.

### Additional Methods

In some embodiments herein, a method further comprises refining a organic molecule to optimize the types, shapes, and sizes of the organic molecule mixture to produce a fuel product. Typical refining processes in the fuel industry include, but are not limited to, distillation, fractionation, extraction, solvent extraction, hydrotreatment, isomerization, dimerization, alkylation, thermal cracking, fluid catalytic cracking, thermofor catalytic cracking, catalytic cracking, steam cracking, and hydrocracking.

In some instances, products generated by a device herein are blended or mixed with fossil fuel or petroleum based feedstocks before refining. For example, a terpene product collected from a device is blended with crude petroleum. In some instances, the petroleum has already been refined before blending with a terpene product. For example, the petroleum is gasoline, diesel, or jet fuel. In other instances, the petroleum is a mixture for fuel blending, for example, a organic molecule mixture that when blended with another organic molecule mixture can create a suitable fuel product.

Provided herein are compositions and methods for creating products from terpenes and creating product from terpenes from biomass. Examples of products include, but are not limited to, fuel products, fragrance products, and insecticide products. A product can be any substance that releases molecularly stored energy. In an embodiment, a product is organic molecules. In another embodiment, a product is a organic molecule. Examples of fuel products include petrochemical products and their precursors and all other substances that may be useful in the petrochemical industry. Fuel products include, for example, petroleum products, and precursors of petroleum, as well as petrochemicals and precursors thereof. In some embodiments, the fuel product is used for generating substances, or materials, useful in the petrochemical industry, including petroleum products and petrochemicals. In some embodiments, the fuel or fuel products are used in a combustor such as a boiler, kiln, dryer or furnace. Other examples of combustors are internal combustion engines such as vehicle engines or generators, including gasoline engines, diesel engines, jet engines, and others. In some embodiments, fuel products are used to produce plastics, resins, fibers, elastomers, lubricants, and gels.

In some embodiments, the fuel products are blended or combined into mixtures to obtain an end product. For example, the fuel products may be blended to form gasoline of various grades, gasoline with or without additives, lubricating oils of various weights and grades, kerosene of various grades, jet fuel, diesel fuel, heating oil, and chemicals for making plastics and other polymers. Compositions of the fuel products described herein may be combined or blended with fuel products produced by other means.

### Genetic Modification of Organisms

In some embodiments of the subject matter provided herein is a photosynthetic organism contains an expression vector comprising a polynucleotide sequence encoding an enzyme of the DOXP pathway or the MVA pathway. In some embodiments, a photosynthetic organism contains an expression vector comprising a polynucleotide sequence encoding an isoprene synthase variant. In some embodiments, a photosynthetic organism expresses isoprene synthase by extrachromosomal modification. In some embodiments, the photosynthetic organism is Physcomitrella patens or Ceratodon purpureus.

In an aspect, provided is a method comprising collecting a volatile organic molecule that has been released from a photosynthetic organism in a gas layer of a biological solar panel array.

In some embodiments, a method herein comprises genetically modifying the photosynthetic organism prior to the growing the organism in the device. For example, the chloroplast or nucleus of the organism may be transformed to generate enzymes that facilitate the production of terpenes. The terpenes can be naturally occurring in the organism or heterologous. In some embodiments, the organisms are genetically modified to upregulate the production of a terpene that naturally occurs. In some embodiments, the organisms are genetically modified to upregulate the production of a terpene that does not naturally occur. For example, a gene encoding an enzyme that generates a terpene through the MVA or MEP pathway can be inserted into the chloroplast or nucleus of the organism. The enzyme is configured to generate a terpene that does not naturally occur in the organism. In this way, the organism is designed to comprise a measurable amount of a large organic molecule that may be useful in the production of a fuel product.

Any of the products described herein can be prepared by transforming an organism to cause the production by such organism of the product. The organism can be photosynthetic prior to or after transformation.

In some embodiments, provided herein is a method to generate a plant containing chloroplasts that are genetically modified to contain a stably integrated polynucleotide (Hager and Bock, Appl. Microbiol. Biotechnol. 54:302-310, 2000). In some embodiments, a photosynthetic organism as described herein can comprise at least one host cell that is modified to generate a product.

In some embodiments, the photosynthetic organisms herein can be transformed to modify the production of a products with an expression vector, for example, to increase production of a products. The products can be naturally or not naturally produced by the organism.

In some embodiments, an expression vector encodes one or more homologous or heterologous nucleotide sequences (derived from the host organism or from a different organism) and/or one or more autologous nucleotide sequences (derived from the same organism) and/or those that encode homologous or heterologous polypeptides. Examples of autologous nucleotide sequences that are transformed into a host cell include isoprenoid synthetic genes, endogenous promoters and 5′ UTRs from the psbA, atpA, or rbcL genes. In some instances, a heterologous sequence is flanked by two autologous sequences or homologous sequences. Homologous sequences are those that have at least 50%, 60%, 70%, 80%, or 90% homology to the sequence in the host cell. In some instances, a homologous sequence is flanked by two autologous sequences. The first and second homologous sequences enable recombination of the heterologous sequence into the genome of the host organism. The first and second homologous sequences can be at least 100, 200, 300, 400, or 500 nucleotides in length.

In some embodiments herein, a method comprises construction of a genetically manipulated strain of photosynthetic organism by involving the transformation with a nucleic acid which encodes a gene of interest, typically an enzyme capable of converting a precursor into a fuel product or precursor of a fuel product. In some embodiments, a transformation introduce nucleic acids into any plastid of the host cell (for example, chloroplast). Transformed cells are typically plated on selective media following introduction of exogenous nucleic acids.

In some embodiments, a recombinant nucleic acid molecule useful in a method herein is contained in a vector. Furthermore, where the method is performed using a second (or more) recombinant nucleic acid molecules, the second recombinant nucleic acid molecule also is contained in a vector, which can, but need not, be the same vector as that containing the first recombinant nucleic acid molecule. In some instance, the vector is any vector useful for introducing a polynucleotide into a chloroplast and; preferably, includes a nucleotide sequence of chloroplast genomic DNA that is sufficient to undergo homologous recombination with chloroplast genomic DNA. Chloroplast vectors and methods for selecting regions of a chloroplast genome for use as a vector are well known (see, for example, Bock, J. Mol. Biol. 312:425-438, 2001; see, also, Staub and Maliga, Plant Cell 4:39-45, 1992; Kavanagh et al., Genetics 152:1111-1122, 1999, each of which is incorporated herein by reference).

In some instances a vector is a promoter. Promoters may come from any source (for example, viral, bacterial, fungal, protist, animal). In some instances, the nucleic acids are inserted into a vector that comprises a promoter of a photosynthetic organism. The promoter can be a promoter for expression in a chloroplast and/or other plastid. In some instances, the nucleic acids are chloroplast based. Examples of promoters contemplated for insertion of any of the nucleic acids herein into the chloroplast include those disclosed in US Application No. 2004/0014174.

In some embodiments, a vector utilized in the practice of a method or process herein comprises one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, sequences that encode a selectable marker, and the like.

In some embodiments, any of the expression vectors herein further comprise a regulatory control sequence. A regulatory control sequence may include for example, promoter(s), operator(s), repressor(s), enhancer(s), transcription termination sequence(s), sequence(s) that regulate translation, or other regulatory control sequence(s) that are compatible with the host cell and control the expression of the nucleic acid molecules. In some cases, a regulatory control sequence includes transcription control sequence(s) that are able to control, modulate, or effect the initiation, elongation, and/or termination of transcription. For example, a regulatory control sequence can increase transcription and translation rate and/or efficiency of a gene or gene product in an organism, wherein expression of the gene or gene product is upregulated resulting (directly or indirectly) in the increased production of a product described herein. The regulatory control sequence may also result in the increase of production of a product by increasing the stability of a gene or gene product. A regulatory control sequence can be autologous or heterologous, and if heterologous, may be homologous. The regulatory control sequence may encode one or more polypeptides which are enzymes that promote expression and production of products. For example, a heterologous regulatory control sequence may be derived from another species of the same genus of the organism and encode a synthase. In another example, an autologous regulatory control sequence can be derived from an organism in which an expression vector is to be expressed.

Various combinations of the regulatory control sequences described herein may be embodied and combined with other features described herein. In some cases, an expression vector comprises one or more regulatory control sequences operatively linked to a nucleotide sequence encoding a polypeptide that effects, for example, upregulates production of a product described herein. In some cases, an expression vector comprises one or more regulatory control sequences operatively linked to a nucleotide sequence encoding a polypeptide that effect, for example, upregulates production of a product.

A polynucleotide or recombinant nucleic acid molecule can be introduced into plant chloroplasts using any method known in the art. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (Potrykus, Ann. Rev. Plant. Physiol. Plant Mol. Biol. 42:205-225, 1991).

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994).

The expression vectors herein can encode polypeptide(s) that promote the production of intermediates, products, precursors, and derivatives of the products described herein. For example, the expression vectors can encode polypeptide(s) that promote the production of intermediates, products, precursors, and derivatives in the isoprenoid pathway.

Terpene precursors are thought to be generated by two pathways. The mevalonate pathway, or HMG-CoA reductase pathway, generates dimethylallyl pyrophosphate (DMAPP) and isopentyl pyrophosphate (IPP), the common C5 precursor for terpenes. The non-mevalonate pathway is an alternative pathway to form DMAPP and IPP. The DMAPP and IPP may be condensed to form geranyl-diphosphate (GPP), or other precursors, such as farnesyl-diphosphate (FPP), geranylgeranyl-diphosphate (GGPP), from which higher isoprenes are formed.

An expression vector herein may encode polypeptide(s) having a role in the mevalonate pathway, such as, for example, thiolase, HMG-CoA synthase, HMG-CoA reductase, mevalonate kinase, phosphomevalonate kinase, and mevalonate-5-pyrophosphate decarboxylase. In other embodiments, the polypeptides are enzymes in the non-mevalonate pathway, such as DOXP synthase, DOXP reductase, 4-diphosphocytidyl-2-C-methyl-D-erythritol synthase, 4-diphophocytidyl-2-C-methyl-D-erythritol kinase, 2-C-methyl-D-erythritol 2,4,-cyclodiphosphate synthase, HMB-PP synthase, HMB-PP reductase, or DOXP reductoisomerase.

In other instances, an expression vector comprises a nucleotide sequence encoding a polypeptide in an isoprenoid pathway, such as, for example, a synthase-encoding sequence. In some embodiments, the synthase is botryococcene synthase, limonene synthase, 1,8 cineole synthase, α-pinene synthase, camphene synthase, (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase.

In some embodiments, the synthase is β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16β-ol synthase, elizabethatriene synthase, sandalol synthase, patchoulol synthase, zinzanol synthase, cedrol synthase, scareol synthase, copalol synthase, or manool synthase.

Pathways utilized for methods described herein may involve enzymes present in the cytosol, in a plastid (for example, chloroplast), or both. Exogenous nucleic acids encoding the enzymes of certain embodiments may be introduced into a host cell, such that the enzyme encoded is active in the cytosol or in a plastid, or both. In some embodiments, a naturally occurring enzyme which is present in one intracellular compartment (for example, in the cytosol) may be expressed in a different intracellular locale (for example, in the chloroplast), or in both the naturally occurring and non-naturally occurring locales following transformation of the host cell.

Chloroplasts are a productive organelle of photosynthetic organisms and a site of large of amounts of protein synthesis. Any of the expression vectors herein may be selectively adapted for chloroplast expression. A number of chloroplast promoters from higher plants have been described in Kung and Lin, Nucleic Acids Res. 13: 7543-7549 (1985). Gene products may be expressed from the expression vector in the chloroplast. Gene products encoded by expression vectors may also be targeted to the chloroplast by chloroplast targeting sequences. For example, targeting an expression vector or the gene product(s) encoded by an expression vector to the chloroplast may further enhance the effects provided by the regulatory control sequences and sequence(s) encoding a protein or peptide that allows or improves production of a fuel molecule.

Various combinations of the chloroplast targeting described herein may be embodied and combined with other features described herein. For example, a nucleotide sequence encoding a terpene synthase may be operably linked to a nucleotide sequence encoding a chloroplast targeting sequence. A host cell may be transformed with an expression vector encoding limonene synthase targeted to the chloroplast, and thus, may produce more limonene synthase as compared to a host cell transformed with an expression vector encoding limonene synthase but not a chloroplast targeting sequence. The increased limonene synthase expression may produce more of the limonene in comparison to the host cell that produces less.

In yet another example, an expression vector comprising a nucleotide sequence encoding an enzyme that produces a product (for example fuel product, fragrance product, insecticide product) not naturally produced by the organism by using precursors that are naturally produced by the organism as substrates, is targeted to the chloroplast. By targeting the enzyme to the chloroplast, production of the product may be increased in comparison to a host cell wherein the enzyme is expressed, but not targeted to the chloroplast. Without being bound by theory, this may be due to increased precursors being produced in the chloroplast and thus, more product may be produced by the enzyme encoded by the introduced nucleotide sequence.

In one aspect, provided is an isolated nucleic acid sequence that encodes a polypeptide that is inserted into any organism as described herein. In some embodiments, an isolated nucleic acid sequence herein encodes a polypeptide with at least about 85% identity to SEQ ID NO. 1. In some embodiments, an isolated nucleic acid sequence herein encodes a polypeptide with at least about 90% identity to SEQ ID NO. 1. In some embodiments, an isolated nucleic acid sequence herein encodes a polypeptide with at least about 95% identity to SEQ ID. 1. In some embodiments, an isolated nucleic acid sequence herein encodes a polypeptide that is substantially identical to SEQ ID NO. 1. In some embodiments, an isolated nucleic acid sequence herein encodes a polypeptide that is identical to SEQ ID NO. 1.

In some embodiments, the polypeptide has activity as an isoprene synthase enzyme.

In some embodiments, an isolated nucleic acid sequence herein encodes an isoprene synthase polypeptide wherein the nucleic acid hybridizes under stringent conditions to SEQ ID NO. 2 or its complement. In some embodiments, the isolated nucleic acid sequence hybridizes under highly stringent conditions.

In some embodiments, an isolated nucleic acid sequence herein is incorporated into the DNA of Physcomitrella patens.

In some embodiments, an isolated nucleic acid sequence herein is incorporated into the DNA of Ceratodon purpureus.

In an aspect, an isolated nucleic acid sequences are provided that encode a polypeptide and are capable of being inserted into an organism as described herein. In some embodiments, an isolated variant of the amino acid sequence of SEQ ID NO. 1 comprises a polypeptide that is at least about 85% identical to SEQ ID NO. 1. In some embodiments, an isolated variant of the amino acid sequence of SEQ ID NO. 1 comprises an polypeptide is at least about 90% identical to SEQ ID NO. 1. In some embodiments, an isolated variant of the amino acid sequence of SEQ ID NO. 1 comprises a polypeptide that is at least about 95% identical to SEQ ID NO. 1.

In an aspect, an isolated polypeptide comprises the amino acid sequence of SEQ ID NO. 1.

In some embodiments, the polypeptide has activity as an isoprene synthase enzyme. In some embodiments, the polypeptide is encoded by Physcomitrella patens or Ceratodon purpureus.

### Example 1

### Product Collection

As provided herein, an example of the methods and systems herein for collection of product as described herein is provided for as the hollow fiber unit 25 of FIG. 4. As shown in FIG. 6, the hollow fiber unit is configured as in a natural gas dehydration arrangement (Air Products PRISM system), see figure below. In this system, the hollow fiber material is hydrophilic, allowing a low concentration of water vapor in natural gas to pass through the membrane, while retaining the organic molecules. While this “normal phase” separation may be preferable in cases where the concentration of the volatile product is substantial, in many cases, the organic molecule content will be low. In such a case, the “reversed phase” separation mode (whereby the organic molecule passes the membrane, and water is retained) is often more efficient. In this case, a hollow fiber composed of silicone (e.g., the PermSelect membrane system offered by MedArray, 3915 Research Park, Drive Suite A-4, Ann Arbor, Mich.) Gases permeate silicone by a solution/diffusion mechanism, whereby the rate of gas permeation is directly proportional to the product of solubility of the gas, and the rate of diffusion of the dissolved gas in silicone. The permeability coefficient is a parameter defined as the transport flux of a gas (rate of gas permeation per unit area), per unit transmembrane driving force, per unit membrane thickness. The rate of gas transfer across the membrane is proportional to the gas permeability coefficient, the membrane surface area, the trans-membrane gas partial pressure difference, and inversely proportional to the membrane thickness. Thus gas transfer across a membrane increases with increased gas permeability coefficient, increased surface area, increased transmembrane gas partial pressure and decreased membrane thickness. The hollow fiber unit thus contains a hydrophobic canister that allows organic molecules to pass through while water vapor is retained. Waste gas is vented through vent 26 while pass-through gas enriched in product and depleted in water vapor is condensed in condenser 27 and collected in tank 28.

Hollow fibers, as demonstrated in the example of FIGS. 6 and 7, constitute a self supported, inherently stable membrane structure that can tolerate high pressure differences between the inside and outside of the hollow fiber. Hollow fibers are typically packaged in membrane modules in which thousands of hollow fibers are bundled in a very compact volume and sealed or potted within a housing as shown in FIG. 7. Consequently, the sum of the surface area of each individual hollow fiber constitutes the total membrane area for the module, and it becomes apparent how it is possible to achieve high membrane surface densities with hollow fibers.

### Example 2

### Genetic Modification of Moss

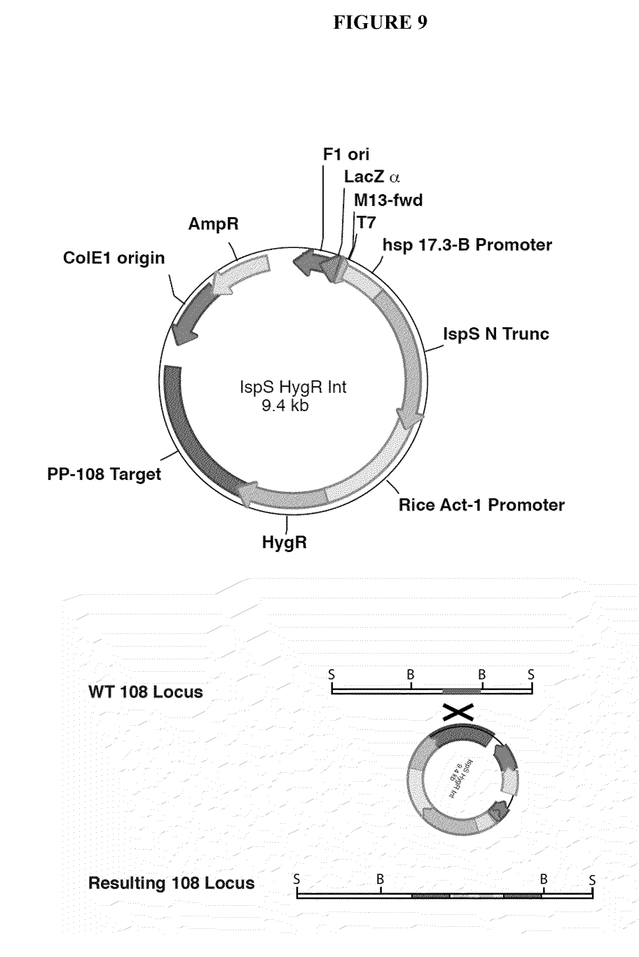

FIG. 9 illustrates an exemplary and non-limiting method of plasmid construction and genetic modification of a moss (for example, Saidi et al Plant Mol Biol 59, 697-711 (2005).) Plasmid IspS HygR Ins is constructed as follows: a temperature-inducible promoter region (−512 to −5 bp) from the soybean hsp17.3-B gene (Schoffl, F., Raschke, E. and Nagao, R. T. The EMBO J. 3: 2491-2497) is cloned as an XbaI-BamHI fragment in pBlueScript KS+ II (Stratagene, La Jolla, Calif.). A synthetic gene encoding sequence ID1 which codes for a catalytically active, C-terminal fragment of Populus alba isoprene synthase, is synthesized as a BamHI fragment by DNA2.0, using GeneDesigner software to approximate the codon frequencies in Physcomitrella patens while eliminating restriction sites that complicate subsequent cloning. This truncation is a modified version of P. alba MEA(+)TEV, which (among others) was identified as having high constitutive activity in Patent Application WO 2009/132220, as a C-terminal extension (His-tag, plus extraneous residues from the cloning vector) and one residue shorter on the N-terminus. Finally, a 4.7 kb KpnI fragment from pBS-BAMH108 vector containing a hygromycin resistance cassette driven by the rice actin-1 gene promoter (McElroy, D., et al., 1991. Mol. Gen. Genet. 231: 150-160) and 1.9 kb of PP-108 genomic locus as targeting fragment (Schaefer, D. G. and Zryd, J. P. 1997. Plant J. 11: 1195-1206.), was inserted in the KpnI restriction site of the vector to create the plasmid used in moss transformation.

Moss transformation is performed following Schaefer, D. G. and Zryd, J. P. 1997. Plant J. 11: 1195-1206, where stable transformants are selected on hygromycin containing media and screened for stable incorporation into the PP-108 site. See, e.g., FIG. 9.

### Example 3

### Manufacture of the Device

Provided herein, in this example, is a device manufacturing process providing for the coating a 4×8×0.03 acryllic (e.g., plastic) sheet with a 3 mm thick layer of 1% agarose containing nutrients, then inoculating this agar layer with plant material. After growth of the plant to confluence, a semipermeable membrane. Mounted on a frame is placed on top (with a spacer and sealed edges). The device, exposed to a light source (sunlight) collects volatiles from the photosynthesis process, which is then isolated by condensation of the atmosphere of the apparatus.

### Example 4

### Manufacture of the Device

Also provided herein is the manufacturing of a device by integrating the moss layer into a pre-existing fabric mesh. In three dimensions, the protonema grow horizontally, to increase the area that the plant covers, while the leafy gametophyte structure grows vertically toward light. This natural tendency is exploited by placing a loose mesh fabric (e.g, Nylon netting #8603, 1/16 in hexagonal holes, available from Christensen NetWorks, 401 Lincoln St. Everson, Wash.) over the protonema phase material and then inducing gametophyte maturation by withdrawing or exhausting the vitamins present in the media.

### Example 5

SEQ ID NO. 1
MTEARRSANYEPNSWDYDYLLSSDTDESIEVYKDKAKKLEAEVRREINNE
KAEFLTLLELIDNVQRLGLGYRFESDIRGALDRFVSSGGFDAVTKTSLHG
TALSFRLLRQHGFEVSQEAFSGFKDQNGNFLENLKEDIKAILSLYEASFL
ALEGENILDEAKVFAISHLKELSEEKIGKELAEQVNHALELPLFIRRTQR
LEAVWSIEAYRKKEDANQVLLELAILDYNMIQSVYQRDLRETSRWWRRVG
LATKLHFARDRLIESFYWAVGVAFEPQYSDCRNSVAKMFSFVTIIDDIYD
VYGTLDELELFTDAVERWDVNAINDLPDYMKLCFLALYNTINEIAYDNLK
DKGENILPYLTKAWADLCNAFLQEAKWLYNKSTPTFDDYFGNAWKSSSGP
LQLVFAYFAVVQNIKKEEIENLQKYHDTISRPSHIFRLCNDLASASAEIA
RGETANSVSCYMRTKGISEELATESVMNLIDETWKKMNKEKLGGSLFAKP
FVETAINLARQSHCTYHNGDAHTSPDELTRKRVLSVITEPILPFER

### Example 6

SEQ ID NO. 2
ggatccATGACTGAAGCTAGACGTTCTGCTAATTATGAGCCTAACTCGTG
GGATTACGATTATCTGTTGTCGTCCGATACCGATGAATCAATCGAGGTGT
ACAAGGATAAGGCTAAGAAATTGGAGGCCGAAGTGAGGAGAGAGATTAAT
AACGAGAAGGCTGAATTCTTGACATTGCTTGAGCTGATTGATAACGTGCA
GCGATTGGGGCTCGGCTATCGTTTTGAGAGCGATATCCGTGGTGCTCTTG
ATCGCTTTGTGTCATCGGGAGGATTCGACGCCGTTACCAAGACGTCTTTG
CACGGAACCGCCCTGAGCTTCAGGCTTCTGAGGCAACACGGGTTTGAGGT
GAGTCAAGAGGCTTTCAGCGGTTTCAAAGATCAGAATGGCAATTTTCTGG
AGAACTTGAAGGAGGATATTAAGGCAATTCTCTCACTTTACGAAGCTAGT
TTCCTTGCTCTGGAAGGTGAGAATATTTTGGACGAAGCTAAGGTGTTTGC
TATTAGTCATCTGAAGGAGCTGTCAGAGGAAAAGATCGGTAAAGAACTTG
CCGAACAGGTAAATCACGCACTCGAACTCCCGCTGCATCGTAGAACGCAG
AGGCTCGAGGCTGTATGGAGTATTGAGGCATACCGCAAGAAGGAAGACGC
TAACCAGGTCCTGCTGGAGCTGGCAATCCTCGACTATAACATGATTCAAA
GCGTGTACCAAAGAGATCTCCGCGAAACGTCACGTTGGTGGCGTCGGGTG
GGGCTCGCCACAAAGTTGCATTTCGCACGCGATCGACTTATTGAATCTTT
CTACTGGGCCGTGGGTGTTGCCTTCGAACCGCAGTACTCTGACTGCCGGA
ATTCGGTGGCCAAGATGTTTAGCTTCGTTACCATTATCGACGATATTTAT
GACGTTTACGGCACATTGGATGAGCTGGAACTGTTTACCGACGCCGTGGA
ACGTTGGGACGTTAACGCAATCAACGACCTGCCCGACTATATGAAACTCT
GCTTCCTGGCACTGTACAACACTATTAATGAGATAGCTTACGATAATCTG
AAGGACAAGGGTGAAAACATCCTCCCATATCTCACCAAGGCTTGGGCCGA
TCTGTGTAACGCATTCCTCCAAGAGGCCAAGTGGCTTTACAATAAATCTA
CCCCGACATTCGATGACTACTTCGGGAACGCATGGAAGAGTTCCTCGGGG
CCCCTCCAACTTGTGTTCGCTTATTTCGCTGTTGTGCAGAACATTAAGAA
GGAGGAGATTGAAAACCTTCAAAAATACCACGATACGATTTCGCGACCTT
CACATATCTTTAGGCTTTGTAACGACCTTGCATCTGCTAGCGCTGAAATC
GCTAGAGGCGAGACTGCCAACAGTGTGTCGTGCTACATGAGGACTAAGGG
TATCAGCGAGGAGCTCGCAACCGAGTCAGTGATGAACTTGATTGACGAAA
CTTGGAAAAAGATGAACAAGGAAAAATTGGGCGGATCCTTGTTCGCTAAG
CCGTTTGTGGAGACAGCCATTAATTTGGCACGGCAATCCCATTGCACGTA
TCATAATGGAGACGCACACACGAGTCCAGATGAACTGACGCGAAAGCGGG
TACTGAGCGTGATTACTGAGCCTATTCTCCCCTTTGAGCGGTAGTAGTAG
ggatcc

What is claimed is:

1. A biological solar panel device comprising:
(a) a layer comprising a material that is transparent or translucent to light;
(b) a photosynthetic layer comprising a material that uses carbon dioxide and water in the presence of light to release a volatile organic molecule, wherein the photosynthetic layer is separated from the transparent or translucent material by a gas layer; and
(c) a layer that provides support for the material that releases a volatile organic molecule.

2. The device of claim 1 wherein the transparent or translucent material comprises an organic polymer.

3. The device of claim 1 wherein the transparent or translucent material comprises a polysilicate material.

4. The device of claim 1 wherein the transparent or translucent material comprises a gas permeable material.

5. The device of claim 1 wherein the transparent or translucent layer allows at least one component of the gas phase to pass through

6. The device of claim 1 wherein the transparent or translucent layer prevents at least one component of the gas phase from passing through

7. The device of claim 1 wherein the transparent or translucent layer material blocks at least some wavelengths of light

8. The device of claim 1 wherein the material that uses carbon dioxide and water in the presence of sunlight to release a volatile organic molecule comprises at least one biochemically-active layer

9. The device of claim 1 wherein the volatile organic molecule comprises at least one terpene or at least one terpenoid.

10. The device of claim 1 wherein the support layer supports the growth of the photosynthetic layer

11. The device of claim 1 wherein the support layer further comprises a feed layer.

12. The device of claim 1 wherein the support layer is porous to nutrients.

13. The device of claim 1 wherein the support layer is substantially impermeable to the volatile organic molecule passing through the support layer.

14. The device of claim 1 wherein the support layer is removable from the device.

15. The device of claim 1 wherein the support layer provides dimensional support to the photosynthetic layer.

16. The device of claim 1 that is connected to at least ten additional biological solar panel devices.

17. The device of claim 1 that is connected to at least twenty-five additional biological solar panel devices.

18. A method comprising collecting a volatile organic molecule that has been released from a material that is in a photosynthetic layer of a biological solar panel device.

19. The method of claim 18 wherein a photosynthetic organism contains an expression construct comprising a polynucleotide sequence encoding at least one enzyme of the DOXP pathway or the MVA pathway.

20. The method of claim 18 wherein a photosynthetic organism contains an expression construct comprising a polynucleotide sequence encoding an isoprene synthase variant.

21. A method comprising inducing synthesis of a volatile organic molecule that uses carbon dioxide and water in the presence of light in a photosynthetic layer of an array of biological solar panel devices wherein the biological solar panel device comprises:
(a) a layer comprising a material is transparent or translucent to light;
(b) the photosynthetic layer comprising the material that uses carbon dioxide and water in the presence of sunlight to release a volatile organic molecule, wherein the photosynthetic layer is separated from the transparent or translucent material by a gas layer; and
(c) a layer that provides support for the material that releases a volatile organic molecule.

22. The method of claim 21 wherein the temperature of the support layer that provides support for immobilizing a photosynthetic organism in the photosynthetic layer is maintained between about 0° C. to about 45° C.

23. The method of claim 21 wherein synthesis of the volatile organic molecule by the photosynthetic layer is induced by adding an inducing substance, removing a nutrient, or exposure to light
